(19) United States
(12) Patent Application Publication
(10) Pub. No.: US 2022/0339556 A1
(43) Pub. Date: Oct. 27, 2022

(54) AUTOMATED FLUIDIC ASSAY BASED ON MOLECULARLY IMPRINTED POLYMER FOR COVID-19 DIAGNOSTICS AND SEROSURVEILLANCE

(71) Applicant: THE ROYAL INSTITUTION FOR THE ADVANCEMENT OF LEARNING/MCGILL UNIVERSITY, Montreal (CA)

(72) Inventors: Sara MAHSHID, Mont-Royal (CA); Roozbeh SIAVASH MOAKHAR, Montréal (CA); Carolina DEL REAL MATA, Montréal (CA)

(21) Appl. No.: 17/730,337

(22) Filed: Apr. 27, 2022

Related U.S. Application Data
(60) Provisional application No. 63/180,327, filed on Apr. 27, 2021.

(51) Int. Cl. B01D 15/38; B01J 20/26

(52) U.S. Cl. CPC B01D 15/3852; B01J 20/268; G01N 2600/00; B82Y 5/00

(57) ABSTRACT

It is provided a biosensor, a device containing same and method for detecting a target protein, the biosensor comprising a nano/micro islands (NMIs) core of gold spatially oriented with nanorough protrusions, and a layer of electropolymerized molecularly imprinted polymers (MIP) polymerized on the NMIs core, said MIP consisting of a conductive monomer comprising a built-in recognition site of the target protein, wherein the MIPs generate an electrical signal upon binding of the target protein.

### CROSS-REFERENCE TO RELATED APPLICATIONS

The present application claims benefit of U.S. Provisional Application No. 63/180,327 filed Apr. 27, 2022, the content of which is herewith incorporated in its entirety.

### TECHNICAL FIELD

It is provided a nanostructured biosensor based on gold nano/micro islands (NMI) for detecting proteins.

### BACKGROUND

The global wave of the coronavirus pandemic has exposed critical gaps in monitoring, and mitigating the spread of respiratory viral infections, highlighting the need for rapid, reliable, and accurate diagnostic tests for the detection of viruses like SARS-CoV-2 and Influeza A at the point-of-need. The existing gold standard detection techniques have failed to satisfy the ongoing need for early-diagnosis and serological testing on demand, being hindered by the need of specialized laboratory equipment and inherent assay limitations, like long turnarounds time in centralized laboratory facilities and lengthy protocols. The effective control of a fast-growing outbreak requires major in-community diagnosis to contain the high transmission potential in early infection stages. An ideal test platform should detect the level of the infection and the body's immunity by targeting both the virus and specific antibodies.

Ceasing the SARS-CoV-2 pandemic requires the application of diagnostic testing on an immense scale. In-community testing could successfully identify cases enabling contact tracing and containment without lockdowns. The speed of testing process could be significantly increased using Point of Care assays that would allow testing to be carried out in remote locations (particularly at homes). Rapid antigen tests have won the competition and are in the hands of individuals, however, the tests using nasal swabs as the targeted sample for detection, are challenged with numerous false results with low reported sensitivities.

Means for an early and accurate diagnosis of SARS-CoV-2 present a great concern due to the substantial transmission potential occurring in the pre-symptomatic phase. Currently, there are many available diagnosis tests including nucleic acid test (NATs), serological tests, antigen, and ancillary (based on personal devices or hospital laboratory tests). NATs are the most used with more than 80 tests approved by the Food and Drugs Agency (FDA). However, the assays requirements hinder the screening and diagnosis of a suspected infected person of less than 24 h or after the acute phase of infection. Despite the overall effectiveness of the diagnostics methods, most are prone to long assay times and high false-negative rates. Moreover, several methods are not suitable for early diagnosis or require access to expensive and highly specialized equipment. Thus, effective diagnosis in resource-limited settings remains a challenge.

The long virus incubation period of up to 14 days can accelerate the spread of the virus, where an average of 5-6 days is expected between incubation and symptom onset, thus prompting the need for early-detection. Further concerns are the new variants that arise with mutations in viral strains over many infections; (e.g. Alpha B.1.1.7, Delta B.1.617.2, Omicron B.1.1.529, amongst others). These variants exhibit increased transmissibility and potential resistance to vaccines and their detection.

To date, the U.S. Food and Drug Administration (FDA) has solely issued approvals for saliva collection kits for at-home self-collection of biofluids that are stabilized in a diluent buffer and shipped to a centralized facility for subsequent sample pre-treatment and testing. When compared to self-administered nasopharyngeal swabs (71%, 4.93 mean log copies. ml⁻¹), the self-collected saliva (81%, 5.58 mean log copies. ml⁻¹) provides higher sensitivity when the diagnosis is performed in the first five to ten days of infection. However, untreated saliva typically requires lengthy pre-treatment protocols negatively affecting its potential as target biofluid.

It is thus still highly desired to be provided with a simple and reliable tool for detecting a viral infection such as COVID-19.

The combination of molecular diagnostic and serological testing on a single platform improves the robustness of the result due to the heterogeneity in disease responses. Testing via molecular diagnostics alone is shown to have a positive predictive agreement from 51.9%-79.2%, likely due to viral load clearance from the upper respiratory tract over time. Meanwhile, a combined approach with tandem serology testing increases the positive detection rate to 98.6%-100%, allowing for more reliable responses during the acute and convalescent phase of infection. In addition, the multiplex detection of IgG and IgM antibodies enables the serosurveillance of current and past infections based on the temporal prevalence of the immunoglobulins in response to infection; sequential seroconversion is confirmed to occur first in IgM followed by IgG in early infection, with waning levels of IgM and high levels of IgG during late infection. Finally, the earliest humoral response after the first week of symptom onset has been shown in antibodies targeting the receptor binding domain (RBD) on the spike protein (37.2% and 38.5% positive samples for IgM-RBD and IgG-RBD, respectively) as opposed to their nucleocapsid (N) antibody counterparts (20.5% and 37.2% for IgM-N and IgG-N, respectively). Nonetheless, the diagnostic community has found it highly challenging to achieve the accuracy for parallel diagnosis and serological testing in one single attempt from the easily accessible body fluids.

### SUMMARY

It is provided a biosensor for detecting a target protein comprising a nano/micro islands (NMIs) core of gold spatially oriented with nanorough protrusions, and a layer of electropolymerized molecularly imprinted polymers (MIP) polymerized on the NMIs core, the MIP consisting of a conductive monomer comprising a built-in recognition site of the target protein, wherein the charge transfer resistance and the impedance of the MIPs change upon binding of the target protein.

In an embodiment, the NMIs are electrodeposited on a conductive glass with a reference electrode of Ag/AgCl and a counter electrode of platinum wire.

In another embodiment, the conductive glass is a tin oxide (ITO) substrate.

In a further embodiment, the conductive monomer is polyaniline (PANI) or o-phenylenediamine (o-PD).

In an additional embodiment, the target protein is an antibody, a viral protein or a heart fatty acid binding protein (H-FABP).

In an embodiment, the antibody is a viral antibody.

In a further embodiment, the viral protein is from SARS-CoV-2 or Influenza.

In an added embodiment, the viral protein is from a SARS-CoV-2 variant.

It is also provided a microfluidic read-out apparatus for detecting a target protein in a subject comprising the biosensor defined herein and microfluidic reader.

In an embodiment, the microfluidic read-out apparatus is a multiplex microfluidic apparatus.

In an additional embodiment, the microfluidic reader allows multiplexing different human samples.

In an embodiment, the apparatus recited herein further comprises a WiFi adapter for transferring the read-out signals from the microfluidic reader to a platform.

In an embodiment, the WiFi adapter is a Bluetooth low energy (BLF) connector.

In another embodiment, the platform is a computer or a smartphone.

It is additionally provided a method of detecting a target protein in a subject comprising the steps of providing a sample from the subject, contacting the sample with a biosensor as defined herein, wherein the presence of the target protein changes the charge transfer resistance and/or impedimetric of the MIPs upon binding of the target protein; and transferring the change in charge transfer resistance or impedimetric signal to a microfluidic reader for transforming the signal into a cyclic voltammetry signal or impedimetric signal, wherein the cyclic voltammetry signal or impedimetric signal indicates the presence of the target protein.

In an embodiment, the subject sample is saliva, plasma, or whole blood.

In a particular embodiment, the subject is a human or an animal.

In another embodiment, the method provided herein further comprises transmitting the cyclic voltammetry signal to a platform.

In an embodiment, the cyclic voltammetry signal or impedimetric signal is transmitted by Wi-Fi to the platform.

In another embodiment, the platform depicts the cyclic voltammetry signal or impedimetric signal in a gauge.

In a further embodiment, the cyclic voltammetry signal or impedimetric signal is transmitted to a computer or a smartphone.

In another embodiment, the cyclic voltammetry signal or impedimetric signal indicates the presence of the target protein in 1 min to 11 min.

### BRIEF DESCRIPTION OF THE DRAWINGS

Reference will now be made to the accompanying drawings.

FIG. 1 (a) illustrates a schematic representation of the use of NMIs/MIPs sensor and the detection of SARS CoV-2 as encompassed herein; (b) multiplexed microfluidic device; (c) real image of the portable electrochemical device and the digitization of the readout.

FIG. 2 illustrates a schematic representation of the MIP biosensor fabrication as described herein. (b)-(c) MIP electron microscopy characterization, (d)-(e) molecular docking characterization.

FIG. 3 illustrates the effect of NMI electrode on current density in (a) and impedance in (b).

FIG. 4(a)-(f) illustrates atomic force microscopy (AFM) evaluation of the MIP sensor after template removal.

FIG. 5 illustrates a schematic representation of the multiplexed microfluidic device fabrication with embedded on-chip electrodes; top-view (left), cross-section-view (right), showing in (a) first step lithography to pattern electrode space in an isolating SiO2 thin film covering the ITO-coated glass wafer; in (b) sequential electron-beam evaporation of ZnO/Cr/Au thin film and second lithography step to etch the thin film with the electrode pattern to obtain isolated conductive RE and CE electrodes; in (c) third step lithography to pattern the multiplex fluidic pattern in an SU-8 photoresist layer; in (d) bottom-up fabrication of gold NMI structures using electrodeposition followed by electropolymerization of o-PD with SARS-CoV-2 SP and antibody binding sites shown in the inset; and in (e) PDMS bonding with the fabricated substrate to encapsulate the device with a punched inlet and outlet for fluid flow.

FIG. 6 illustrates the electrochemical characterization of NMI/MIP assay at (a) each step of fabrication ((1) electrodeposition of the enhanced gold NMIs, (2) electropolymerization of the nonconductive o-PD polymer, (3) template removal from the MIPs, (4) target binding) for both SP and antibodies and their (b) cyclic voltammetry, (c) current signal, (d) Bode EIS response, and (e) impedance at 0.1 Hz responses. The electrochemical measurements were done with 5 mM [Fe(CN)6]3-/4- in PBS.

FIG. 7 illustrates the assay incubation time study; the impedimetric signal at different incubation times for SARS-CoV-2 (a) SP in human saliva at 1000 pg. ml-1, (b) IgG-RBD in undiluted human plasma at 100 pg. μl-1, and (c) IgM-RBD in undiluted human plasma at 100 pg. μl-1; (d) optimization of the washing step in template removal to achieve the optimal number of binding sites.

FIG. 8 illustrates the Analytical sensitivity performance metrics; whein in a) the impedimetric signal transduction of SARS-CoV-2 SP on NMI/MIP electrode imprinted with the spike protein (SP) head domain compared with NMI/NIP (non-imprinted polymer electrode) in buffer (black) and saliva (cyan); in (b) the impedance magnitude of SARS-CoV-2 SP on NMI/MIP electrode in different concentrations in saliva and buffer, the correlated linear relation of the impedimetric signal as a function of SP concentration in saliva and buffer belonging to (c) original strain, Alpha B.1.1.7, Delta B.1.617.2, and Omicron B.1.1.529 variants and (d) heat-inactivated SARS-CoV-2 viral particles.

FIG. 9 illustrates the sensitivity of variant spike proteins through bode plots of the impedance magnitude over a relevant range of 10 pg. ml⁻¹-10⁵ pg. ml⁻¹ for the Alpha B.1.1.7 variant SP in (a) buffer and (b) saliva, the Delta B.1.617.2 variant SP in (c) buffer and (d) saliva, and the Omicron B.1.1.529 variant SP in (e) buffer and (f) saliva.

FIG. 10 Bode plots for impedimetric detection of heat-inactivated SARS-CoV-2 viral particles in (a) buffer and (b) saliva from 9.60′10³-3.84′10⁸ number of viral particles. ml⁻¹; (c) selectivity study over the linear range of SP detection in saliva and buffer at 1000 pg. ml⁻¹, *** p<0.001.

FIG. 11 illustrates the calibration plots for impedimetric serology study in buffer, plasma, and whole blood of (a) IgG-RBD, (b) IgG-N and (c) corresponding calibration curve; calibration plots for serology in buffer, plasma, and whole blood of (d) IgM-RBD, (e) IgM-N and (f) corresponding calibration curve; targeted range from 10 pg. μl⁻¹-10⁴ pg. μl⁻¹. Comparable impedimetric responses were shown for both anti-RBD (IgG-RBD and IgM-RBD) and anti-nucleocapsid (IgG-N and IgM-N) antibodies in each of the three biofluids, demonstrating the functionality of the assay independent of the biofluid selection. Marginally higher impedimetric responses for whole blood are likely due to the presence of interferent molecules and blood cells, but the electrochemical response remains within a comparable range of impedance magnitude.

FIG. 12 illustrates the analytical performance metrics of antibodies for SARS-CoV2 detection (a) The impedance magnitude of SARS-CoV-2 IgG-RBD and IgM-RBD antibodies on NMI/MIP electrode in whole blood. The correlated linear relation of the impedimetric signal as a function of (b) IgG-RBD and (c) IgM-RBD concentration in whole blood, plasma, and buffer. Data shows mean values±standard deviation (n=3).

FIG. 13 illustrates the analytical selectivity performance metrics, showing in (a) quantification of the cross-reactivity of the SARS-CoV-2 SP as opposed to different viral SPs. Quantification of the cross-reactivity of the SARS-CoV-2 antibodies in (b) IgG-RBD and in (c) IgM-RBD compared with different viral IgG and IgM antibodies, *** p<0.001. Data shows mean values±standard deviation (n=3). In (d) Cross reactivity study of SP assays for different variants: original strain, alpha, delta, and omicron SPs at 100 pg. ml⁻¹. The SPs of variants were tested on the original strain nano-imprinted polymer assay, which demonstrated the ability to detect even minute changes in protein morphology. Although the original strain NMI/MIP assay can detect a positive result for emerging variants, the noticeable signal drop indicated the high resolution of the MIPs that can differentiate structural refinements based on single amino acid mutations.

FIG. 14 illustrates the selectivity study of over the linear range of antibody detection in blood, plasma, and buffer for (a) IgG-RBD and (b) IgM-RBD at 50 pg. μl⁻¹. Cross reactivity study for (c) IgG-N on the IgG-RBD imprinted assay (F_{1,4}=305.487, p=6.29139E-5), (d) IgM-N on the IgM-RBD imprinted assay (F_{1,4}=119.298, p=3.99023E-4), (e) IgG-RBD belonging to the Delta variant on the IgG-RBD original strain imprinted assay showing non-significant response due to similarity of the binding affinities between antigens and antibodies (F_{1,4}=0.28315, p=0.62283); at 100 pg. μl⁻¹, *** p<0.001.

FIG. 15 illustrates the bode plots of raw impedance response detecting whole viral particles, in (a) original stains and in (b) Delta variant.

FIG. 16 the bode plots of raw impedance response detecting IgG-RBD, IgG-N, IgM-RBD and IgM-N, from patient whole blood samples diagnosed with (a) the original strain of SARS-CoV-2 and (b) Delta variant of SARS-CoV-2. And from patient undiluted plasma samples diagnosed with (c) the original strain of SARS-CoV-2 and (d) Delta variant of SARS-CoV-2.

FIG. 17 illustrates the assessment of quantitative multiplexed NFluidEX test assay for the detection of SARS-CoV-2 in saliva and blood samples using COVID-19-positive and -negative subjects, showing in (a) the impedimetric signal analysis of saliva samples comparing healthy and patient signals, with inset (i) null comparison demonstrating the distinguished signal level in patients with COVID-19-positive compared to healthy controls (F_{1,122}=161.77, p=4.11E-24), and inset (ii) the ROC curve showing 100% sensing efficiency; in (b) the serosurveillance of IgG-RBD from COVID-19-positive and -negative subjects with the inset showing statistical analysis of the impedimetric signal average level in patient whole blood (F_{1,27}=261.78, p=2.03E-15) and plasma samples (F_{1,27}=105.05, p=8.34E-11) compared to healthy controls, *** p<0.001. In (c) the serosurveillance of IgM-RBD from COVID-19-positive and -negative subjects with an inset statistical analysis of the impedimetric signal average level in patient whole blood (F_{1,36}=48.61, p=3.57E-8) and plasma samples (F_{1,36}=73.19, p=3.37E-10) compared to healthy controls, *** p<0.001. In (d) quantitative correlation of NFluidEX impedimetric signal from COVID-19-positive samples with the test assay calibration curve based on viral particle load. In (e) the linear regression and 95% confidence intervals to compare the statistical significance of the NFluidEX quantitative response with RT-qPCR. In (f) case study of NFluidEX response for 10 patients using both the diagnosis and serology tests. In (g) quantitative response of the NFluidEX case study compared with RT-qPCR Ct values. Data shows mean values±standard deviation (n=3).

FIG. 18 NMI/MIP assay fabrication. (a) Gold NMIs electrodeposition using chronoamperometry, (b) corresponding charge on the working electrode during electrodeposition becoming more negative, (c) cyclic voltammetry for o-PD electropolymerization.

FIG. 19 illustrates the MIP assay fabricated for Influenza detection. Bode response showing the impedance magnitude for the detection of Influenza spike protein (a) buffer and (b) saliva, and (c) corresponding linear calibration plot for Influenza spike protein. Bode response showing the impedance magnitude in for the detection of heat-inactivated Influenza viral particles in (d) buffer and (e) saliva, and (f) corresponding linear calibration plot for heat-inactivated Influenza viral particles.

FIG. 20 illustrates in detail the layout of the printed circuit board modified for NFluidEX testing. Top view of PCB labelled with primary electronic components.

FIG. 21 illustrates an schematic representation of the interior assembly of the electrochemical device for portable point-of-care testing. The NFIuidEX potentiostat design included an updated Bluetooth protocol (Bluetooth Low Energy 5.1) and a relay module to allow multiplexed detection for both serological test assays (IgG and IgM) and diagnostic assay (whole virus via SP). In (a) a schematic of open housing unit labelled with primary electrochemical components. In (b) a close-up view of alignment between the microfluidic test strip and the SPE adaptors embedded into the side port of the potentiostat housing unit. In (c) a close-up view of switch setup for multiplexed analysis. A manual switch allows for user-mediated switching between blood or saliva sample mode. In (d) a top view of test strip, detailing its compartments for multiplexed sensing. In (e) a block diagram of potentiostat network with circuit schematic of the multiplexed switch setup. Working electrodes of the IgG and IgM testing assays are connected to the NO (Normally Open) and NC (Normally Closed) ports of the relay, which rapidly flips the AC voltage between them to perform pseudo-simultaneous measurements. Real image of the smartphone gadget for SARS-CoV-2 risk-assessment showing in (f) the real-time potentiostat, a sample collection cartridge and multiplexing microfluidic enclosing the NMIs/MIPs; aside the smart-phone gadget is designed to receive the cyclic voltammetry signal using Wi-Fi, process the signal, and depict the risk-level in a color-blind friendly gauge; and in d) the smart-phone gadget indicates the stage if the infection based on cyclic voltammetry.

FIG. 22 illustrate the digitization of the EIS readout signal. To perform the electrochemical measurements, an EIS smartphone application was designed (based on the open-source Android application that communicated with a Bluetooth Low Energy (BLE) module; set parameters include the sample per frequency decade as 1, DC bias voltage as 0, signal amplitude as 0.01 V, and electrode configuration as ‘3-electrode’. In (a) a flow chart of software interface to perform EIS measurements in different test assays. In (b) a decision chart with threshold values based on three times the standard deviation of the highest healthy samples. In (c) (top) the results of the saliva diagnostic test for whole viral particle presence, (middle) the results of the serological blood test for antibody presence, and (bottom) the results of the overall test with combined diagnostic and serology testing.

FIG. 23 illustrate sample collection cartridge, showing in (a) the design of the removable sample collection kit that combines sample collection, pre-treatment, and microfluidic flow on a single apparatus, in (b) a perspective view of the sample collection cartridge with labelled components; a saliva capture funnel for direct self-collection of saliva, a blood collection window that exposes the inlet of the blood microchannel to the finger prick blood from the user, a single-release trigger that is used to press down on the PDMS soft lithography buttons (only when the trigger is removed, the buttons will be lifted to enable the suction-based flow), in (c) real image of 3D-printed cartridge with inserted electrochemical microfluidic device and corresponding dimensions, and in (d) a sectional view of the sample collection workflow and point-of-care automated biofluid flow.

### DETAILED DESCRIPTION

In accordance with the present invention, there is provided a biosensor for detecting a target protein comprising a nano/micro islands (NMIs) core of gold spatially oriented with nanorough protrusions, and a layer of electropolymerized molecularly imprinted polymers (MIP) polymerized on the NMIs core, said MIP consisting of a conductive monomer comprising a built-in recognition site of the target protein, wherein the MIPs generate an electrical signal upon binding of the target protein.

It is provided a portable, rapid, quantitative, and inexpensive diagnostic and serological home-test kit analogous to a glucometer for sensitive detection of SARS-CoV-2 viral particles and SARS-CoV-2 nucleocapsid and spike antibody at the point-of- need, in particular at home and in remote locations.

The tool provided herein address three challenges: (1) detecting the presence of SARS-CoV-2 and emerging variants at the early-stages of the infection from easily accessible body fluids such as saliva, (2) detecting the antibodies in response to the infection from whole blood, and (3) monitoring the efficacy of therapy once the patient is under treatment by quantifying both SARS-CoV-2 spike proteins (SP) and specific antibodies.

The kit integrates a new biomimetic receptor based on molecularly imprinted polymers (MIP), a new nanostructured sensor based on gold nano/micro islands (NMI) and a portable microfluidic-impedimetric read-out. As encompassed herein, the microfluidic read-out apparatus can be a multiplex microfluidic apparatus. When the virus or the antibody binds to the NMIs/MIPs morphological-detection site, a signal is generated. The NMIs/MIPs microfluidic device achieves a rapid detection in 10 min for the SARS-CoV-2 whole virus in human saliva and SARS-CoV-2 antibodies in undiluted plasma and 1 min in whole blood. Also, the device presents a high specificity towards SARS-CoV-2 over Influenza A virus. In an embodiment, it is encompassed that the present device has a high specificity towards the severe acute respiratory syndrome coronavirus 1 (SARS-CoV-1), the human coronavirus 229E (HCoV-229E), or the Middle East respiratory syndrome coronavirus (MERS-CoV). The detection system is integrated with a WiFi adapter to send the readout signals to a smartphone. The WiFi adapter can be for e.g. but not limited to a Bluetooth low energy (BLF) connector The signals are analyzed in a user-friendly smartphone software, and the level of SARS-CoV-2 virus or SARS-CoV-2 antibody is visualized for nonprofessional users.

MIPs consists of a synthetic polymer with recognitions sites build-up from specific templates through a co-polymerization of functional monomers and cross-linkers evolving a template of the target. Templates species are needed for the generation of recognition sites during the polymerization process. After the template removal, the imprinted polymer can be used for a rebinding step, where the target, present in a solution can be recognized by the blank binding site built-in synthetic polymer matrix. From low molecular weight molecules to micro-organisms MIPs have proven their value as sensors for many different biological applications. MIPs offer the possibility of a sensitive and highly selective biorecognition sensor based solely on synthetic materials, avoiding the need for fragile biomolecules. The main advantages of MIPs are their cost-effectiveness, easy to scale production, improved shelf-life, stability, and versatility. Recently, the development of MIPs for virus detection has attracted attention due to its versatility and capability to be designed to detect the whole virus or specific proteins. The binding of the target on the MIPs changes the electrical properties (charge transfer resistance) of the polymer, which is measured with a cyclic voltammetry signal and/or impedimetric signal.

However, MIPs' known low sensitivity towards the detection of some proteins and biological compounds have hindered their use for highly sensitive applications.

It is provided in an embodiment a method for rapid detection of SARS-CoV2 (whole virus) and spike protein antibodies in biological fluids. The innovative part of the assay is a new biomimetic receptor based on highly selective molecularly imprinted polymer (MIP) assay combined with nano/micro islands (NMIs) of gold with spatial orientation and nanorough protrusions NMIs to form a core-shell structure. The gold NMIs, microfluidics, the biomimetic receptor based on MIP and a portable electrical (impedimetric) read-out are provided for sensitive and quantitative detection of SARS-CoV2 and antibodies in human saliva and human whole blood, respectively. In an embodiment, the bio receptor is based on a conductive monomer, which is electropolymerized in the presence of a target (here heat-inactivated SARS-CoV2 and spike protein antibodies). The target will be removed to leave a template, which can rebind to the target and generate an electrical signal. The NMIs enhances the electropolymerizing of the MIPs, overcoming MIPs challenges and offering a highly sensitive and selective technique for an electrochemical readout system. The important part of the MIP assay is optimizing the electropolymerization and determining the type of polymer. Thus, different polymers were evaluated to optimize the sensitivity and selectivity of the assay. The size of the heat-inactivated SARS-CoV-2 and SARS-CoV-2 Abs are about 200-500 nm and 10 nm, respectively. Therefore, polyaniline (PANI) uses whole virus as template and both spike protein and Abs are done with a thin polymer, o-phenylenediamine (o-PD). Further, the electropolymerization process was optimized in terms of polymer concentration, number of deposition cycles and acidity of the solution and by comparing the electrocatalytic activity and charge transfer resistance of formed polymer layers. The NMIs/MIPs sensor were characterized via cyclic voltammetry (CV) and electrochemical impedance spectroscopy (EIS) at every step of the fabrication to complete the NMIs/MIPs optimization. Afterwards, the NMIs/MIPs sensor was tested with real samples achieving a rapid detection of the target in 10-15 minutes. The NMIs/MIPs for SARS-CoV-2 presents a low limit of detection (LOD) for SARS-CoV-2 spike protein (Original strain: 5.89 pg.ml⁻¹; Alpha variant: 6.48 pg.ml⁻¹; Delta variant: 8.13 pg.ml⁻¹; Omicron variant: 7.62 pg.ml⁻¹ in human saliva with a linear range of 10 pg ml⁻¹-10⁵ pg. ml⁻¹. Moreover, it shows a high specificity towards detection of SARS-CoV-2, severe acute respiratory syndrome coronavirus 1 (SARS-CoV-1), the human coronavirus 229E (HCoV-229E), the Middle East respiratory syndrome coronavirus (MERS-CoV) vs. Influenza A H1N1. The NMIs/MIPs serological approach for detection of the spike protein antibody was tested in whole blood reaching a LOD of 3.13 pg. μl⁻¹ with a linear range of 10 pg. μl⁻¹-10⁴ pg. All LODs for original variant, SP and Abs are detailed in Table 1.

TABLE 1
Limit of detection and linear range of NFluidEX for
various targets in saliva, plasma, blood, and buffer.

| Target | Biofluid | Limit of detection | | Linear range | |
|---|---|---|---|---|---|
| Original strain SP | Saliva | 5.89 | pg · ml⁻¹ | 1e1-1e5 | pg · ml⁻¹ |
| | Buffer | 3.79 | pg · ml⁻¹ | | |
| Alpha variant SP | Saliva | 6.48 | pg · ml⁻¹ | | |
| | Buffer | 4.51 | pg · ml⁻¹ | | |
| Delta variant SP | Saliva | 8.13 | pg · ml⁻¹ | | |
| | Buffer | 6.28 | pg · ml⁻¹ | | |
| Omicron variant SP | Saliva | 7.62 | pg · ml⁻¹ | | |
| | Buffer | 4.72 | pg · ml⁻¹ | | |
| Heat-inactivated | Saliva | 948.4 number of viral | | 9.60e3-3.84e8 number of | |
| SARS-CoV-2 viral | | particles · ml⁻¹ | | viral particles · ml⁻¹ | |
| particles | Buffer | 2091.6 number of viral | | | |
| | | particles · ml⁻¹ | | | |
| IgG-RBD | Plasma | 4.06 | pg · ml⁻¹ | 1e1-1e4 | pg · ml⁻¹ |
| | Blood | 5.74 | pg · ml⁻¹ | | |
| | Buffer | 3.63 | pg · ml⁻¹ | | |
| IgM-RBD | Plasma | 2.97 | pg · ml⁻¹ | | |
| | Blood | 3.13 | pg · ml⁻¹ | | |
| | Buffer | 2.79 | pg · ml⁻¹ | | |
| IgG-N | Plasma | 6.94 | pg · ml⁻¹ | | |
| | Blood | 7.76 | pg · ml⁻¹ | | |
| | Buffer | 5.18 | pg · ml⁻¹ | | |
| IgM-N | Plasma | 3.25 | pg · ml⁻¹ | | |
| | Blood | 3.58 | pg · ml⁻¹ | | |
| | Buffer | 2.99 | g · ml⁻¹ | | |
| Influenza A SP | Saliva | 8.63 | pg · ml⁻¹ | 1e1-1e5 | pg · ml⁻¹ |
| | Buffer | 3.99 | pg · ml⁻¹ | | |

| Heat-inactivated | Saliva | 2,576,415 number of | 6.44e6-2.58e9 number of |
|---|---|---|---|
| Influenza A viral | | viral particles · ml⁻¹ | viral particles · ml⁻¹ |
| particles | Buffer | 1,105,422 number of | |
| | | viral particles · ml⁻¹ | |

The adaptability nature of viruses presents a challenge to the gold standard diagnosis. The proposed assay offers a versatile approach for the fabrication of the biomimetic receptor that can be tuned towards different targets based on their physical and morphological characteristics. This unique aspects of the proposed MIP assay will bypass the need for in-depth knowledge of the virus mutations and chemistry and can be easily adapted for future applications, such as quasi-simultaneus detection of Abs in blood and SP in saliva samples.

The proposed microfluidic device integrating the proposed biomimetic electrochemical sensor based on a NMIs/MIPs system with facilitated electropolymerization and enhanced signal read-out, provided a fast, portable, stable, achieved a simple system ultrasensitive and selectivity as an alternative diagnostic test for detection of SARS-CoV2.

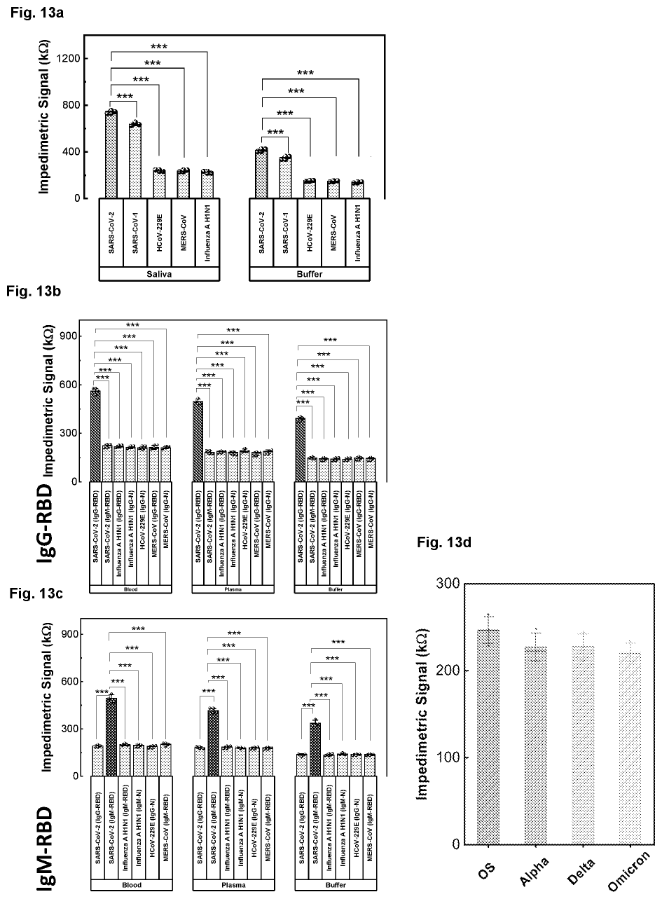

It is encompassed that the device provided herein allows detection of any proteins, not just viruses such as SARS-CoV2 (see FIG. 13).

The integration of a new biomimetic receptor based on MIP with a hierarchical gold nanostructure in the form of nano/micro islands (NMIs) with spatial orientation and nanorough protrusions creates a core-shell structure composed of the NMIs (the core) and a thin layer of electropolymerized MIP (the shell) with enhanced sensitivity and selectivity for the detection of SARS-CoV2 whole virus and antibodies. The bio receptor is based on a conductive monomer, which is electropolymerized in the presence of a target (e.g. heat-inactivated SARS-CoV2 and spike protein antibodies). The target will be removed to leave a template, which can rebind to the target and generate an electrical signal. The gold NMIs will provide a large surface area for immobilization of the MIP and enhancement of the sensitivity.

The MIPs/NMI sensor demonstrates a rapid, sensitive, and selective electrochemical response in a broad range of dilution of SARS-CoV2 and antibodies in saliva and whole blood respectively, confirming its presence in biological fluids.

The sensor provided herein allows for a rapid response since the NMIs/MIPs time for an optimal incubation was determined to be 10-15 min, for human saliva. This time is highly reduced in comparison to the golden standard qRT-PCR test which takes approximately 4-6 hours to provide results. The exemplified NMIs/MIPs are demonstrated to be highly sensitive for SARS-CoV-2 microfluidic device, with a low limit of detection (LOD) for SARS-CoV-2 spike protein of: original strain: 5.89 pg.ml⁻¹; Alpha variant: 6.48 pg.ml⁻¹; Delta variant: 8.13 pg.ml⁻¹; Omicron variant: 7.62 pg.ml⁻¹l in human saliva with a linear range of 10 pg. ml⁻¹-10⁵ pg. ml⁻¹. Additionally, the NMIs/MIPs for the spike protein antibody was tested in undiluted human plasma reaching a LOD of 3.13 pg. μl⁻¹ with a linear range of 10 pg. μl⁻¹-10⁴ pg. By having both assays in one device for example, e.g. a multiplex microfluidic device, the disease stage limitations presented by a qRT-PCR and serological test can be overcome. The golden standard qRT-PCR is limited to the acute phase of infection, while the serological test is limited to later stages of the disease. The device described herein combines both approaches to overcome these limitations. The device/process is showed to be highly selectivity and specific, as demonstrated by effectively detecting SARS-CoV-2 over highly similar viral load as is Influenza A virus, and also including severe acute respiratory syndrome coronavirus 1 (SARS-CoV-1), the human coronavirus 229E (HCoV-229E), the Middle East respiratory syndrome coronavirus (MERS-CoV), thus avoiding possible cross-reactivity with other coronaviruses.

The electrochemical NMIs/MIPs sensor do not require highly trained technicians nor expensive equipment. It only require a PC/cellphone reader and potentiostat. In that sense is superior to qRT-PCR which requires expensive instrumentation and highly trained laboratory personnel, proficient in performing the test. Moreover, NMIs/MIPs sensor has a simple and straightforward operation since is integrated into a sample delivery microfluidic system to improve the control of conditions and throughput, allowing for multiplexing. Contrary to qRT-PCR, which requires extended sample preparation in addition to the test protocol, the microfluidic device sample preparation is simple. The sensor effectively detects the target via CV and EIS test. A digitalized system for the analysis of the cyclic voltammetry readout is provided to couple with a PC/phone friendly platform.

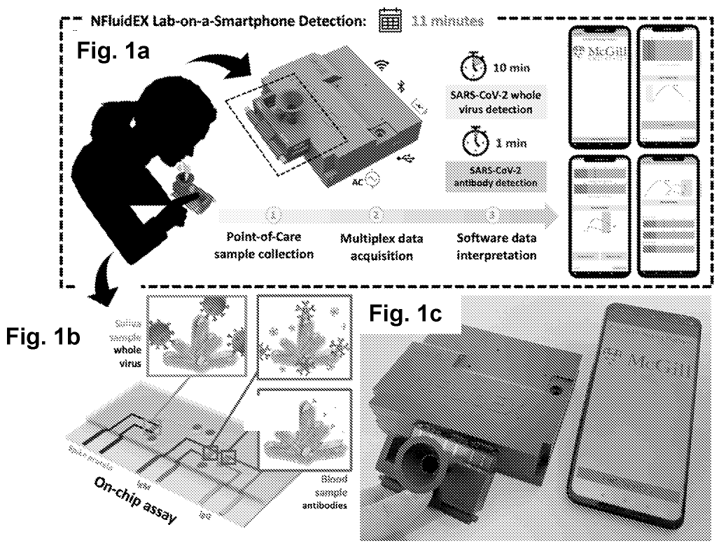

A stepwise schematic of the NMIs/MIPs sensor and the detection of SARS CoV-2 through the whole virus and/or a specific antibody is presented in FIG. 1. The biomimetic microfluidic sensor provided herein is based on the synergic combination of MIP with an NMIs core structure (NMIs/MIPs) for the electrochemical detection of SARS-CoV-2 virus 30 and SARS-CoV-2 nucleocapsid and spike antibody 32 present in the saliva 11 or blood 12 of a patient 10. The MIPs/NMIs sensor 20 is embedded in a microfluidic which increase the control of the system for the fast detection of SARS-CoV-2 in a portable fashion. Additionally, a digitalized system 13 for the analysis of the cyclic voltammetry readout is provided to couple with a PC/phone friendly platform 15. The readout can be performed qualitatively on a technical interface 16 from a technician (doctor/physician, nurse) perspective, or on a friendly patient interface 17.

The device electrochemical performance is studied, via cyclic voltammetry (CV) and electrochemical impedance spectroscopy (EIS), in the presence of two different targets, the heat-inactivated SARS-CoV-2 whole virus 30 or its nucleocapsid or spike protein antibody 32. NMIs provide an ideal extended and surface area and robust core for the NMIs/MIPs fabrication. Specifically, the NMIs/MIPs sensor was tested in a controlled environment with Heat inactivated SARS CoV-2 virus spiked in 1×PBS (7.2 pH) and in real media conditions, for which the heat-inactivated virus was spiked in saliva from healthy donors. Moreover, the NMIs/MIPs sensor for nucleocapsid and spike protein antibody detection was effectively tested in a 1×PBS (7.2 pH) buffer and in two human body fluids, undiluted plasma, and whole blood from healthy donors. Unlike other technique which are functional solely in diluted plasma media, the assay can successfully detect SARS-CoV-2 antibody in undiluted human plasma and in addition to this can detect antibodies in whole blood.

The tool provided synergistically combine nanostructured gold sensors with microfluidic sample delivery systems and biomolecular assay capabilities. The proposed device is based on a new hierarchical gold nanostructured platform in the form of nano/micro islands (NMIs) with spatial orientation and nanorough protrusions. It is known that gold NMIs enabled sensitive and quantifiable detection of bacteria (E. Coli, MRSA etc.) and small molecules (e.g. H-FABP protein). The gold NMIs, microfluidics, the new biomimetic receptor based on molecularly imprinted polymers (MIP), and the portable electrical (impedimetric) read-out were harnessed for sensitive and quantitative detection of SARS-CoV-2 viral particles and SARS-CoV-2 nucleocapsid and spike antibodies, analogous to a glucometer. MIPs report physical stability, structure predictability, specificity, versatility, simple fabrication, and cost-effectiveness

The MIP recognition element is based on a thin layer of a conductive monomer, which is electropolymerized in the presence of a target template (whole virus, viral protein, spike antibody, or nucleocapsid antibody). The template will be removed to leave a built-in recognition site, which can rebind to the target and generate an electrical signal. The tens of nanometer thickness of the film ensure a partial coverage of the template molecule, in turn, easing the diffusion of the target analyte. The highly sensitive and selective MIP biorecognition approach is a suitable synthetic alternative for effective detection of SARS-CoV-2. Recently development of custom-made MIPs for whole virus or specific viral proteins detection has grown, expanding MIP's capabilities for accurate binding of viral sub-types.

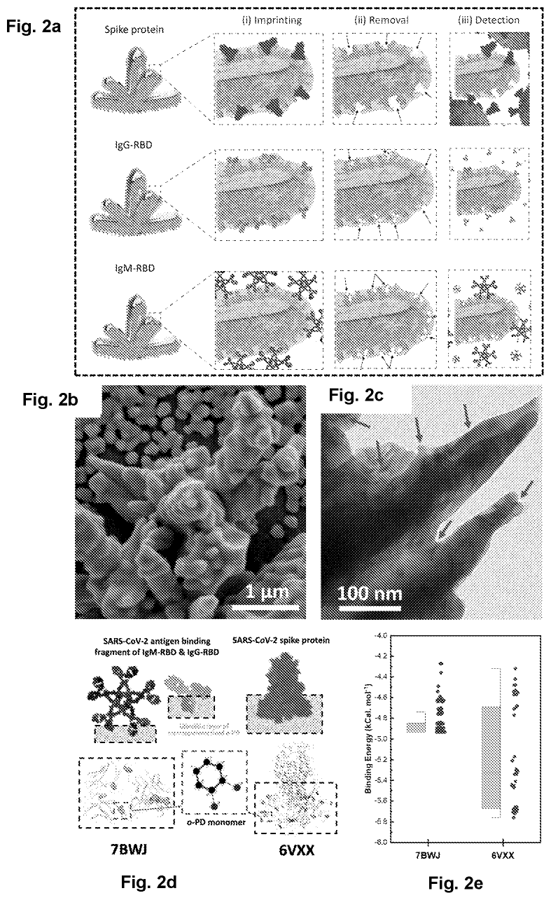

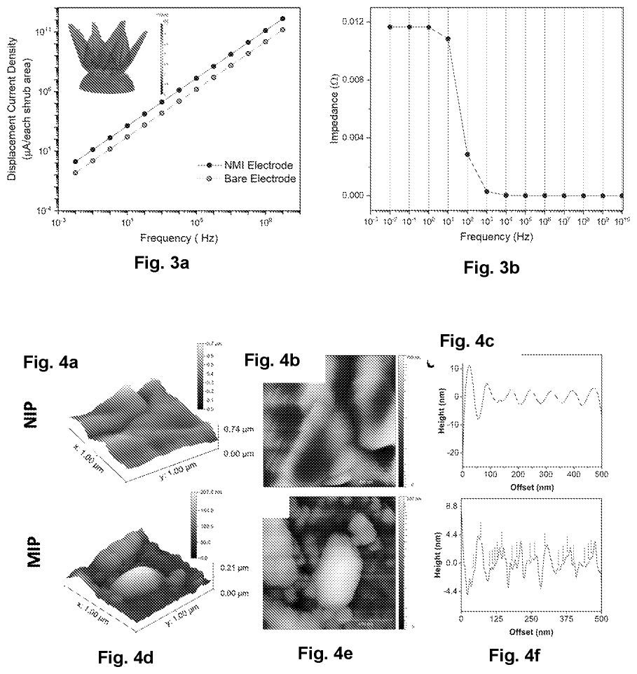

Briefly, the MIP biosensor are fabricated through electrodeposition of gold MNIs 22 on a indium tin oxide (ITO) substrate 20 where the analysis wells are patterned through standard lithography (see FIG. 5), followed by an aniline MIP layer electropolymerized in the presence of Heat-Inactivated SARS-CoV-2 as the required template 24 or o-PD in presence of SARS-CoV-2 nucleocapsid and spike antibody 26. Last, the template is removed through a wash step 28. The MIP biosensor are then able to detect SARS-CoV-2 whole virus 30 or its nucleocapsid or spike protein antibody 32. In an alternative, the MIP biosensor can be fabricated with o-PD MIP layer electropolymerized in the presence of spike protein of SARS-CoV-2 or SARS-CoV-2 nucleocapsid and spike antibody 26, as the required template 24. The sensitivity of the biomimic NMI/MIP assay strongly depends on the structural configuration and enhanced electroactivity of NMI structures. The test chamber design confers a reproducible and ˜6 times enhanced electrochemical signal transduction response arising from the uniform distribution of the large surface area micro dimensional gold NMI surface (FIG. 2b). The electrochemically induced defects and dislocations (arrows in FIG. 2c) The electrochemical patterning of the surface with NMIs gives rise to a theoretically enhanced localized electrical field confinement at the edges of the nano-protuberance for enhanced electrocatalytic activity at the surface of the electrode. The appendage microstructure grown on the conductive base that transfers a concentrated electric field, is enhanced locally along the edges and nano-protuberances, which gives rise to a collective plasmon oscillation. The enhanced localized electric field expedites the charge transference by reducing the electron transfer barriers and consequently, enhances the signal transduction that positively affects the detection sensitivity for low traces of the target analyte. In the nano-protuberance of a single NMI enhance the signal sensitivity through elevated aspect ratio geometries, structural isotropy, entropy, and surface energy. A finite element method simulation was performed using COMSOL multiphysiscs results showed that current density was increased more than five times by adding NMI structures (FIG. 3), which was likely due to the higher geometric aspect ratio and isotropy of the protrusion surface. The sharp edges of the NMI electrodes provided a steep electric field gradient, which enabled a higher electrical current. Also, the NMI structures provided 10 times higher surface area (28.9 μm² versus 2.82 μm²) for a high surface-to-volume ratio, resulting in predictably enhanced electrochemical biosensing. (FIG. 3a) Observing the corresponding impedance of the simulated electrode over the frequency sweep demonstrated the highest impedimetric response for low frequencies values, particularly at 0, 0.1 and 0.01 Hz (FIG. 3b). As such, the most sensitive response was expected to occur over these low probing frequencies.

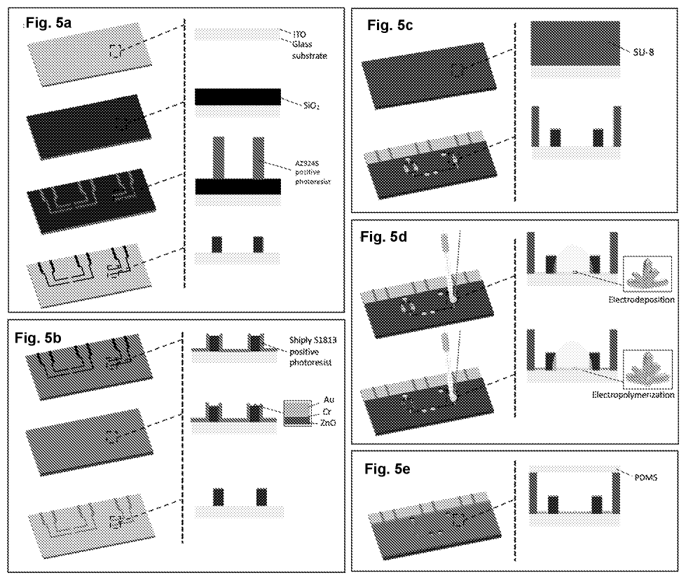

The selectivity of the biomimic NMI/MIP recognition assay relies on the formation of binding sites in the polymeric thin films imprinted by SP, IgG-RBD, or IgM-RBD that is central to selective recognition in saliva and blood, respectively. We used a thin layer (5-10 nm) of o-PD polymer to interact with SP and antibody entities of the virus to record their spatial molecular configuration. We benchmarked the proficiency of templating the polymer layer with the virus entities by investigating the affinity of the antigen-binding fragment of the IgG-RBD and IgM-RBD (7BWJ) and the SP (6VXX) from Protein Data Bank (PDB), with the o-PD polymer layer using molecular docking (MD) simulations to determine the most favourable binding sites (FIG. 2d). The amino acids on the template protein conjugate with o-PD to form recognition sites and to impart chemical functionality into the binding pockets. These preferential binding sites are determined based on binding energy quantification (FIG. 2e). For the 6VXX SP, preferential binding of the o-PD monomers is primarily in the head region near the RBD, thereby demonstrating monomer competition for stabilizing interactions in a region with a high binding affinity of −5.22 kcal. mol⁻¹. Similarly, quantification of free energy minimization for the 7BWJ antigen-binding fragment showed competitive stabilization with a binding affinity of −4.85 kcal. mol⁻¹. The directional interactions of o-PD monomers with the head region binding domain of the proteins allow for partial confinement of proteins in the polymer, which is essential for their removal to form the empty geometrical shapes in polymer for selective detection. Formation of the selective geometrical pockets and template protein removal is confirmed by 3D surface topography (FIG. 4a), demonstrating a rough and indented surface casted with respect to the size and shape of the template proteins for the selective nano-imprinted geometric sites, in contrast to (FIG. 4b) the 2D profile of the NIP electrode. FIGS. 4c, 4d, 4e and 4f correspond the one-dimensional nanoroughness of the NIP electrode, the 3D topology of the MIP electrode, the 2D profile of the MIP electrode, the one-dimensional nanoroughness of the MIP electrode with orange arrows showing the imprinted recognition sites, respectively.

The ideal properties in the materials use for the construction of electrodes depend on their application. In this work, a highly sensitive detection of SARS-CoV-2 virus and SARS-CoV-2 antibodies is desired on a stable linear range. Thus, aiming for a material with increased surface area, high electrical conductivity and of facile fabrication. In this work the conductive glass “ITO” is selected for the base of the electrode. An ITO glass-coated wafer while a single-step lithography was utilized to pattern the fluidic channels. Initially the ITO-coated glass was deposited with a 5 mm silicon dioxide insulating layer using plasma-enhanced chemical vapor deposition (PECVD) at a deposition rate of 10 nm. s⁻¹. Then the electrochemical reference electrode (RE) and counter electrode (CE) were patterned in an AZ9245 photoresist (10 μm) followed by etching the patterned electrodes in the SiO₂ via BOE etching (FIG. 5a). A thin-film consisting of zinc oxide (60 nm), chromium (10 nm), and gold (200 nm) was deposited via electron-beam deposition (BJD 1600) followed by the second lithography step to pattern the electrode in a Shipley photoresist with a thickness of 2.14 μm. A wet etching step using HF was utilized to remove the un-patterned gold thin-film and develop well isolated conductive gold electrodes upon photoresist lift-off (FIG. 5b) with a final RE and CE dimensions of 7.7 mm long and 1.76 mm wide, 5.9 mm spaced apart (compatibility with the adapters assembled in the PCB). Next, a tertiary lithography step was used to fabricate the multiplex fluidic channels in an SU-8 layer with a thickness of 50 μm aligning the sensing chamber over the electrochemical electrodes (FIG. 5c). Further deposited with gold for its well-stablished electrical properties, stability and biocompatibility. The increment of surface area was achieved through Mahshid Lab's protocol for the electrodeposition of 3D gold NMIs (FIG. 5d). The unique morphology of the nanosized shrub provide an ideal extended and surface area and robust core for the NMIs/MIPs fabrication. Aniline, a low conductive monomer, was selected to produce thin films. The generation of open binding sites due to only partial coverage of the target molecule have been reported to ease the diffusion of the target analyte into them. Moreover, the non-conductive ortho-phenylenediamine (o-PD), which generates ultrathin monomer films, is ideal for detection of protein-sized targets. Finally, a PDMS layer with the same size as the wafer (57 mm×24 mm) was bonded to the wafer to encapsulate the channels via plasma treatment (FIG. 5e).

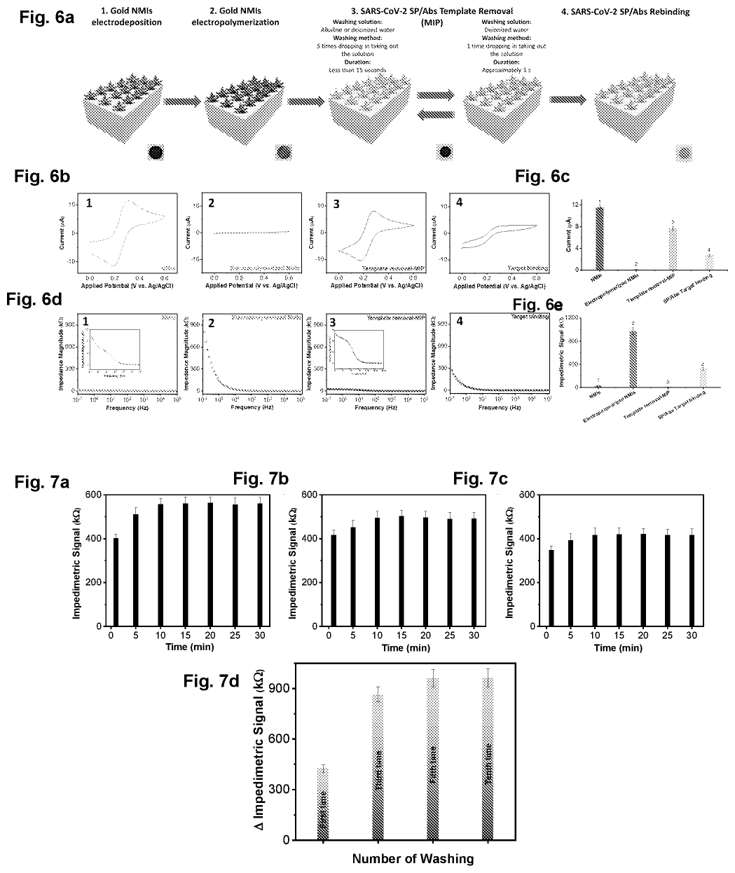

The electrochemical characterization after each step of the electrode fabrication is shown in FIG. 6. The steps as observed in FIG. 6a: (1) electrodeposition of the enhanced gold NMIs, (2) electropolymerization of the nonconductive o-PD polymer, (3) template removal from the MIPs, (4) target binding. FIG. 6b show the cyclic voltammetry responses at each step; the redox peak for NMIs is sharply suppressed in the presence of a nonconductive polymer to confirm the coverage with o-PD during electropolymerization, (FIG. 6c) the current signal at 0.3 V at each fabrication step, (FIG. 6d) the Bode EIS response at each step, and (FIG. 6e) the corresponding impedance signal at 0.1 Hz for each step; the highest sensitivity of the assay is in the low frequency range, with the greatest difference in impedimetric response at 0.1 Hz; After the electropolymerization, the current is decreased, and impedance magnitude is increased due to complete coverage of the electrode surface with nonconductive o-PD. Template removal shows an increase in current confirming the partial coverage. A measurable drop in the current and consequent increase in impedance magnitude is resulted from target binding to the surface.

The NMI/MIP enables a multiplex parallel analysis of saliva samples for whole virus detection via its SP, and blood samples for serology testing of IgG-RBD and IgM-RBD antibodies. The analytical assessment of the NMIs/MIPs is evaluated based on the impedance magnitude within the biological concentration ranges (ng. ml⁻¹ to μg. ml⁻¹) in buffer solution and in body fluids including saliva, plasma, and blood. All signal recordings were obtained by a potentiostat/galvanostat module with a potential amplitude of 10 mV. The working principle of the NMI/MIP signal transduction is based on the detection of an increase in impedance magnitude of the spectroscopic signal upon interaction of the electrodes with the specific domains of viral SP or antibodies in the designated chambers. An incubation time of 10 min for viral SPs in saliva and 1 min for IgG-RBD and IgM-RBD in whole blood is required for the optimal performance of the assays (FIG. 7). A period of 10-min is considered as the optimal incubation time as negligible differences were observed after 10-min incubation (FIG. 7a). For the whole blood, the incubation period was determined to be 1 min to prevent coagulation on the surface of the electrode (FIG. 7b-c). Additionally, the impedance magnitude differences with respect to the electropolymerized NMIs after various number of washing repeats with ethanol and water (5:1) and 0.1 M NaOH (optimal washing solution). After five times washing, most of the template proteins were removed from the structure (FIG. 7d).

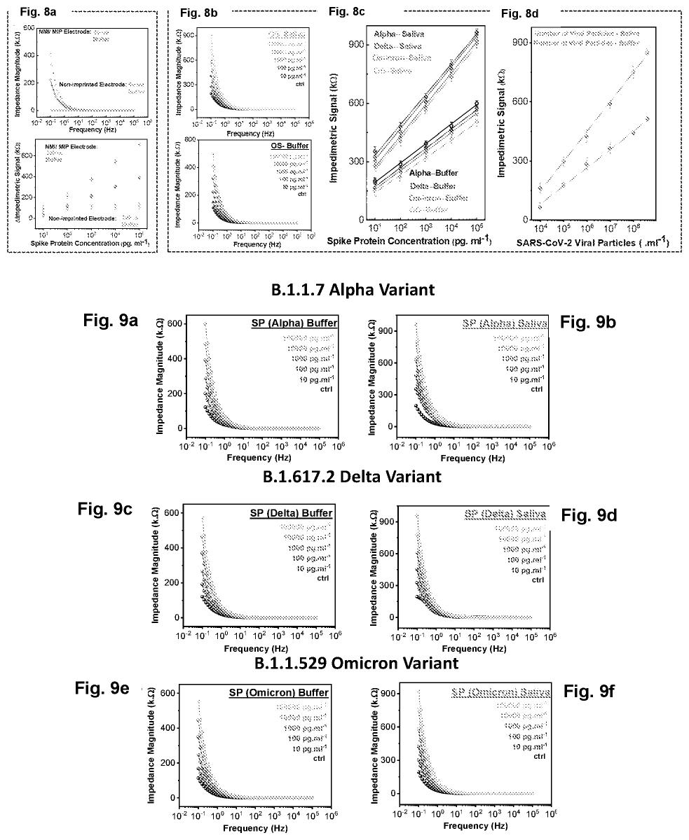

The sensitive signal transduction at a low concentration of SARS-CoV-2 (10 pg. ml⁻¹) is achieved due to the NMI/MIP electrodes harbouring the binding sites. This is evident by comparing the impedimetric signal of SARS-CoV-2 viral SP on the biomimic NMI/MIP test assay with imprinted SP binding sites, with that of NMI/non-imprinted polymer (NIP) electrode (without imprinted binding sites) (FIG. 8a). The negligible impedimetric magnitude changes in both buffer and human saliva on the NMI/NIP electrode confirm the lack of imprinted binding sites in the nonconductive o-PD layer compared to the NMI/MIP.

In both buffer and saliva media, and for all tested concentrations, the NMI/MIP electrodes bearing the binding sites generate a sensitive differentiable signal. For the SP, the increase in the impedance signal was positively correlated to the increase in the concentration of the virus (FIG. 8b and FIG. 9). The frequency in which the signal transduction is performed, tremendously affects the signal resolution at low concentrations of analytes (0.1-10⁵ Hz). As such, when extenuating the test frequency to 0.1 Hz, which led to an increased signal resolution capable of fully distinguishing concentrations as low as 10 pg. ml⁻¹. The impedimetric signal increased linearly with the concentration of the SARS-CoV-2 SP within the range of 10 pg. ml⁻¹-10⁵ pg. ml⁻¹ for the original strain Alpha B.1.1.7, Delta B.1.617.2, and Omicron B.1.1.529 variants, after a 10-min incubation time (FIG. 6c). Similarly, spiked saliva samples demonstrated a linear signal behavior with respect to increase of concentration (R²=0.99), with negligible interference of saliva media. The calculated LOD of the NFluidEX is in the low pg. ml⁻¹ ranges for the detection of SARS-CoV-2 SP (Original strain: 5.89 pg.ml⁻¹; Alpha variant: 6.48 pg.ml⁻¹; Delta variant: 8.13 pg.ml⁻¹; Omicron variant: 7.62 pg.ml⁻¹; IgG-RBD and IgM-RBD: 3-7 pg.μl⁻¹), and a wide linear range (10 pg. ml⁻¹-10⁵ pg. ml⁻¹) (Table 2 and 3) stands out when compared to other literature reports and COVID-19 Emergency Use Authorization (EUA) medical devices, particularly with its superior application in testing the untreated saliva in a short time-window (Table 4-6).

TABLE 2
Comparative table for detection of SARS-CoV-2 virus and antibodies

| Electrodes | Media | Time | | Limit of Detection | | Linear Range | |
|---|---|---|---|---|---|---|---|
| Magnetic bead-based | Untreated saliva | 30 | min | 19 ng/mL (Spike | | — | |
| immunosensor combined with | | | | protein) | | | |
| carbon black-modified screen- | | | | 8 ng/mL (Nucleocapsid | | | |
| printed electrode | | | | protein) | | | |
| GO-Modified with SP RBD | Clinical sera | 30 | min | 1 | ng/mL | — | |
| inmobilized and SKI bloqued | | | | | | | |
| p-sulfocalix[8]arene (SCX8) | Various clinical specimens | Not stated | | 200 | copies/mL | — | |
| functionalized graphene | | | | | | | |
| (SCX8-RGO) | | | | | | | |
| GO-8H-EDC-NHS-Au NS | Blood, saliva and | 1 | min | 1.68 × 10⁻²² | μg/mL | — | |
| | oropharyngeal/nasopharyngeal | | | | | | |
| | swab | | | | | | |
| Au-thin film electrode (TFE) - | Lysis buffer | 15 | min | 15 | fM | 2.22-111 | fM |
| interfaced with a MIP from | | | | | | | |
| (poly-m-phenylenediamine | | | | | | | |
| (PmPD)) | | | | | | | |
| Capture probe-conjugated | Processed nasopharyngeal | <2 | hours | 1 | copy/μL | 1 to 1 × 109 | copies/μL |
| magnetic bead particle | swab sample | | | | | | |
| (CP-MNB) | | | | | | | |
| Tethered Au nanostructurated | Unprocessed saliva | 5 | min | 4 × 10³ | particles/mL | — | |
| bearing an analyte-binding | | | | | | | |
| antibody | | | | | | | |
| Gold NMIs/PANI | Unprocessed saliva | 10 | min | 5.89 | pg · ml⁻¹ | 1e1-1e5 | pg · ml⁻¹ |
| Gold NMIs/o-PD | Whole blood | 1 | min | 3.13 | pg · μl⁻¹ | 1e1-1e4 | pg · μl⁻¹ |

TABLE 3
Limit of detection and linear range of NFluidEX for
various targets in saliva, plasma, blood and buffer

| Target | Biofluid | Limit of detection | | Linear range | |
|---|---|---|---|---|---|
| Original strain SP | Saliva | 5.89 | pg · ml⁻¹ | 1e1-1e5 | pg · ml⁻¹ |
| | Buffer | 3.79 | pg · ml⁻¹ | | |
| Alpha variant SP | Saliva | 6.48 | pg · ml⁻¹ | | |
| | Buffer | 4.51 | pg · ml⁻¹ | | |
| Delta variant SP | Saliva | 8.13 | pg · ml⁻¹ | | |
| | Buffer | 6.28 | pg · ml⁻¹ | | |
| Omicron variant SP | Saliva | 7.62 | pg · ml⁻¹ | | |
| | Buffer | 4.72 | pg · ml⁻¹ | | |
| Heat-inactivated | Saliva | 948.4 number of | | 9.60e3-3.84e8 number of | |
| SARS-CoV-2 viral | | viral particles · ml⁻¹ | | viral particles · ml⁻¹ | |
| particles | Buffer | 2091.6 number of | | | |
| | | viral particles · ml⁻¹ | | | |
| IgG-RBD | Plasma | 4.06 | pg · μl⁻¹ | 1e1-1e4 | pg · μl⁻¹ |
| | Blood | 5.74 | pg · μl⁻¹ | | |
| | Buffer | 3.63 | pg · μl⁻¹ | | |
| IgM-RBD | Plasma | 2.97 | pg · μl⁻¹ | | |
| | Blood | 3.13 | pg · μl⁻¹ | | |
| | Buffer | 2.79 | pg · μl⁻¹ | | |
| IgG-N | Plasma | 6.94 | pg · μl⁻¹ | | |
| | Blood | 7.76 | pg · μl⁻¹ | | |
| | Buffer | 5.18 | pg · μl⁻¹ | | |
| IgM-N | Plasma | 3.25 | pg · μl⁻¹ | | |
| | Blood | 3.58 | pg · μl⁻¹ | | |
| | Buffer | 2.99 | g · μl⁻¹ | | |
| Influenza A SP | Saliva | 8.63 | pg · ml⁻¹ | 1e1-1e5 | pg · ml⁻¹ |
| | Buffer | 3.99 | pg · ml⁻¹ | | |

TABLE 4
Comparative table for detection of SARS-CoV-2 antigen and antibodies.

| | | | | | | Portable | Without |
|---|---|---|---|---|---|---|---|
| | | | | | | signal | Reference |
| SARS-CoV-2 Tests | Media | Time | | Limit of Detection | Linear Range | transduction | Measure |
| Magnetic bead-based | Untreated | 30 | min | SP: 1.9e4 pg · ml⁻¹ | SP: 1.9e4-1e7 pg · ml⁻¹ | Yes | Yes |
| immunosensor | saliva, buffer | | | | | | |
| combined with carbon | | | | | | | |
| black-modified screen- | | | | | | | |
| printed electrode | | | | | | | |
| ePAD paper-based | Clinical sera | 30 | min | SP: 110 pg · ml⁻¹ | SP: 1000-1000e3 pg · ml⁻¹ | No | No |
| sensor: GO-Modified | | | | IgG: 0.96 pg · ml⁻¹ | IgG and IgM: 1-1000 pg · ml⁻¹ | | |
| with SP RBD | | | | IgM: 0.14 pg · ml⁻¹ | | | |
| immobilized and SKI | | | | | | | |
| bloqued | | | | | | | |
| NanoSystem: GO-8H- | Blood, saliva | 1 | min | SP: 1.68e−16 pg · ml⁻¹ | SP: 1-10e−11 pg · ml⁻¹ | No | Yes |
| EDC-NHS-Au NS | and nasal | | | | | | |
| | swab | | | | | | |
| Tethered Au | Unprocessed | 5 | min | SP: 1 pg · ml⁻¹ | SP: 1-100 pg · ml⁻¹ | No | Yes |
| nanostructurated | saliva | | | | | | |
| bearing an analyte- | | | | | | | |
| binding antibody | | | | | | | |
| SPEEDS: | Patient | 13 | min | IgG-S: 10.1 pg · ml⁻¹ | IgG-S: 10.1-6e4 pg · ml⁻¹ | Yes | Yes |
| electrochemical | serum | | | IgM-S: 1.64 pg · ml⁻¹ | IgM-S: 1.64-5e4 pg · ml⁻¹ | | |
| immunosensor | | | | | | | |
| SARS-CoV-2 | Serum and | 1 | min | IgG-S: 250 pg · ml⁻¹ | IgG-S: 2e4-4e4 pg · ml⁻¹ (serum), | Yes | No |
| RapidPlex: Laser | saliva | | | IgM-S: 250 pg · ml⁻¹ | 200-500 pg · ml⁻¹ (saliva) | | |
| engraved graphene | | | | | IgM-S: 2e4-5e4 pg · ml⁻¹ (serum), | | |
| electrodes | | | | | 600-500 pg · ml⁻¹ (saliva) | | |
| Electrochemical | Serum and | Not | | SP: 760 pg · ml⁻¹ | SP: 760-760e3 pg · ml⁻¹ | No | No |
| aptamer-based sensor | artificial | stated | | | | | |
| | saliva | | | | | | |
| Low-cost | Saliva | 6.5 | min | SP and Alpha variant: | SP and Alpha variant: | No | No |
| Electrochemical | | | | 0.229 pg · ml⁻¹ | 0.1-1e3 pg · ml⁻¹ | | |
| Advanced Diagnostic | | | | | | | |
| (LEAD): modified | | | | | | | |
| graphite leads | | | | | | | |
| Carbon nanotube field- | Saliva and | 2-3 | min | SP: 4.12e−3 pg · ml⁻¹ | SP: 0.1e−3-5.0 pg · ml⁻¹ | No | No |
| effect transistor | buffer | | | | | | |
| DSA1N5-Cov-eChip: | 1:1 diluted | 10 | min | Original strain: 0.438 pg · ml⁻¹ | Original, Alpha and Delta variants: | Yes | No |
| aptamer functionalized | saliva | | | Alpha variant: 1.227 pg · ml⁻¹ | 1.752-1927.2 pg · ml⁻¹ | | |
| to gold electrodes | | | | Delta variant: 1.578 pg · ml⁻¹ | | | |
| KAUSTat AuNPs-LSG | Nasal swab | 1 | min | SP, Alpha, Beta and Delta variants: | SP, Alpha, Beta and Delta variants: | Yes | No |
| sensor | | | | 5140 pg · ml⁻¹ | 1e3-500e3 pg · ml⁻¹ | | |
| Flexible organic | Buffer, | 5 | min | IgG: 1.5e−4 pg · ml⁻¹ (buffer), | IgG: 1.5e−3-1.5e3 pg · ml⁻¹ | Yes | No |
| electrochemical | serum, saliva | | | 1.5e−3 pg · ml⁻¹ (saliva, serum) | | | |
| transistors | | | | | | | |
| NFluidEX: NMI/MIP | Untreated | 11 | min | See Table 3 | See Table 3 | Yes | Yes |
| assay | saliva | | | | | | |
| | Whole | | | | | | |
| | blood | | | | | | |

TABLE 5
Comparative table of SARS-CoV-2 FDA EUA Approved Antigen Diagnostic Tests

| | | Sampling | | | | Response |
|---|---|---|---|---|---|---|
| Company | Test | method | PPA | NPA | LOD | time |
| Abbott | Panbio COVID-19 | Nasal swab | 98.1% | 99.8% | 2.5 × 10^{1.8} | 15-20 |
| | Ag Rapid Test | | | | TCID₅₀ · ml⁻¹ | minutes |
| | Device | | | | | |
| Access Bio | CareStart COVID- | Nasal swab | 87% | 98% | 2.8 × 10³ | 10 |
| Inc. | 19 Antigen Home | | | | TCID₅₀ · ml⁻¹ | minutes |
| | Test | | | | | |
| OraSure | InteliSwab COVID- | Nasal swab | 84% | 98% | 2.5 × 10² | 30 |
| Technologies | 19 Rapid Test Rx | | | | TCID₅₀ · ml⁻¹ | minutes |
| Inc. | | | | | | |
| Lumira | LumiraDx SARS- | Nasal swab | 97.6% | 96.6% | 32 | 12 |
| | CoV-2 Ag Test | | | | TCID₅₀ · ml⁻¹ | minutes |
| BTNX Inc. | Rapid Response | Nasal swab | 94.55% | 100% | 2 × 10^{2.4} | 15 |
| | COVID-19 | | | | TCID₅₀ · ml⁻¹ | minutes |
| NFluidEX | NMI/MIP assay | Saliva | 100% | 100% | 14 | 11 |
| | | | | | TCID₅₀ · ml⁻¹ | minutes |

TABLE 6
Comparative table of SARS-CoV-2 FDA EUA Approved Serology Tests

| | | Detected | | | | | Response | |
|---|---|---|---|---|---|---|---|---|
| Company | Test | Antibodies | PPA | NPA | LOD | | time | |
| Access Bio, | Access Bio | IgG-S, IgM-S, | 98.4% | 98.9% | Not stated | | 10 | minutes |
| Inc. | CareStart | IgG-N and | (combined) | (combined) | | | | |
| | COVID-19 | IgM-N | | | | | | |
| | IgM/IgG | | | | | | | |
| Abbott | AdviseDx | IgG-S | 98.1% | 99.6% | ~8.67 | pg · ml⁻¹ | Not stated | |
| | SARS- | | | | | | | |
| | CoV-2 IgG | | | | | | | |
| | II (Alinity) | | | | | | | |
| Siemens | Atellica IM | IgG-S | 100% | 99.9% | ~0.84 | pg · ml⁻¹ | 2 h, 1 min | |
| | SARS- | | | | | | batch | |
| | CoV-2 IgG | | | | | | testing | |
| | (COV2G) | | | | | | | |
| Kantaro | COVID- | IgG-S | 99.15% | 99.6% | ~3.14 | pg · ml⁻¹ | 30 | min |

| | | | | | | | |
|---|---|---|---|---|---|---|---|
| Biosciences | SeroKlir | | | | | | |
| NFluidEX | NMI/MIP | IgG-RBD, IgM-RBD, | 100% | 100% | 2.79-7.76 pg · ml⁻¹ | 11 | min |
| | assay | IgG-N, IgM-N | | | for IgG-RBD, | | |
| | | | | | IgG-N, IgM-N | | |
| | | | | | and IgM-RBD | | |

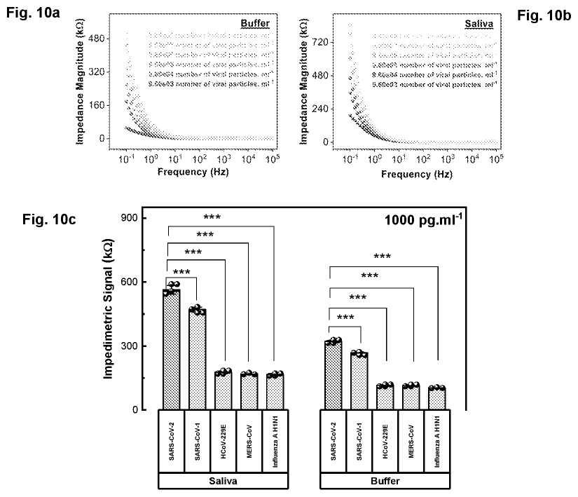

Translating the performance of the test assay based on viral load compared to the concentration of other viral entities is advantageous by enabling detection without lysis, isolation or concentration of the entities. To assess the biomimic NMI/MIP test assay for the detection of whole viral particles, we calibrated the impedimetric signal based on the tested heat-inactivated SARS-CoV-2 particles in physiological concentrations in both buffer and saliva (FIG. 10a-b). The imprinted polymer assay remained amendable over a wide linear range from 9.60×10³-3.84×10⁸ number of viral particles. ml⁻¹ (FIG. 8d), which is comparable with physiologically relevant viral loads in saliva.

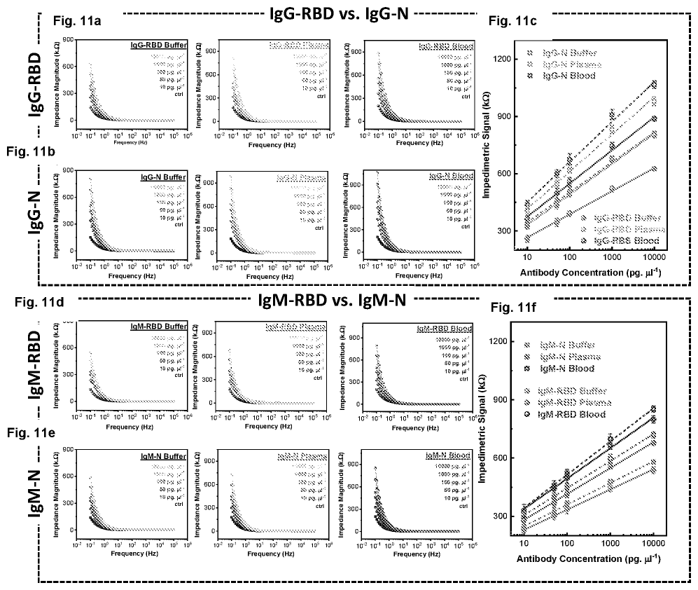

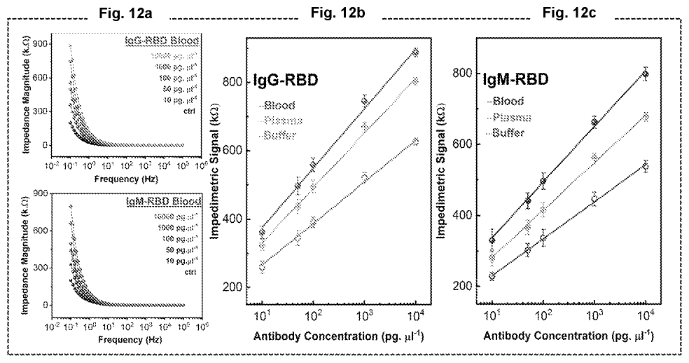

In parallel, the NFluidEX was tested for the detection of both IgG-RBD and IgM-RBD antibodies to determine the ability of the device in serology testing. In the designated chambers, the biomimic NMIs/MIPs were specifically fabricated for the serological detection of SARS-CoV-2 specific antibodies.The impedimetric signal of antibodies in the concentration range of 10 pg.μl⁻¹-10⁴ pg. μl⁻¹ was assessed in spiked buffer, undiluted human plasma, and whole blood, demonstrating an increasing trend with respect to the concentration of IgG-RBD and IgM-RBD (FIG. 12a). A linear relationship between the impedimetric signal and logarithm value of the SARS-CoV-2 antibody concentration over the range of 10 pg. μl⁻¹-10⁴ pg. μl⁻¹ was found in buffer, undiluted human plasma, and whole blood, respectively (FIG. 12b-c, FIG. 11).

To explore the efficacy of NFluidEX for selective detection of SARS-CoV-2 SP in saliva, and both IgG-RBD and IgM-RBD in blood, we obtained the impedimetric signal of SARS-CoV-2 in buffer and saliva compared to the signals of other viral infections that can interfere with SARS-CoV-2 detection due to similarities in shape, size and molecular composition. These include the severe acute respiratory syndrome coronavirus 1 (SARS-CoV-1), the human coronavirus 229E (HCoV-229E), the Middle East respiratory syndrome coronavirus (MERS-CoV), and Influenza A H1N1.

The impedimetric signal from biomimic NMIs/MIPs assay in the NFluidEX device imprinted with SARS-CoV-2 SP demonstrated a higher value towards its matching protein (SARS-CoV-2 SP) compared to the SPs from other tested viruses (FIG. 13a). A null comparison was performed between the results using one-way analysis of variance (ANOVA) with post hoc Holm-Sidak's test. ANOVA demonstrated an overall significant difference among the target SARS-CoV-2 SP in the NMI/MIP test assay and other viral SPs (p<0.001, Table 7), suggesting a low non-specific binding of other viral SPs with the biomimic NMIs/MIPs imprinted with SARS-CoV-2 SP. The selectivity was tested at a lower concentration within the linear range of detection (FIG. 10c) rendering similar selective signal magnitude, which demonstrates that over the concentration range, the presence of the signal of SARS-CoV-2 is still significantly higher than the others. This indicates low cross-reactivity of other viral SPs with the NFluidEX saliva test assay. Notably, SARS-CoV-1 was detectable yet distinguishable on the SARS-CoV-2 SP imprinted assay, indicating that a past virus with high sequence and structural homology will unsurprisingly bind the assay; similar cross-reactivity has been reported on both proposed and commercial clinical tests.

TABLE 7
A summary of statistical significance evaluation using one-way ANOVA with post hoc
Holm-Sidak mean comparison test for the diagnostic selectivity of SARS-CoV-2 SP

| | Saliva p value | | | Buffer p value | | | |
|---|---|---|---|---|---|---|---|
| Mean | 10 | 1000 | 10000 | 10 | 1000 | 10000 | |
| Comparisons | pg · ml⁻¹ | pg · ml⁻¹ | pg · ml⁻¹ | pg · ml⁻¹ | pg · ml⁻¹ | pg · ml⁻¹ | Sig.^{a} |
| Influenza A H1N1 | 3.55E−11 | 4.02E−12 | 3.79E−14 | 1.79E−14 | 2.26E−14 | 1.75E−13 | 1 |
| SARS-CoV-2 | | | | | | | |
| HCoV-229E | 4.58E−11 | 4.44E−12 | 4.69E−14 | 2.23E−14 | 2.96E−14 | 2.10E−13 | 1 |
| SARS-CoV-2 | | | | | | | |
| MERS-CoV | 4.69E−11 | 5.33E−12 | 4.93E−14 | 2.33E−14 | 3.01E−14 | 2.22E−13 | 1 |
| SARS-CoV-2 | | | | | | | |
| Influenza A H1N1 | 2.91E−10 | 5.70E−11 | 3.57E−13 | 1.70E−13 | 2.60E−13 | 1.56E−12 | 1 |
| SARS-CoV-1 | | | | | | | |
| HCoV-229E | 3.98E−10 | 6.50E−11 | 4.65E−13 | 2.24E−13 | 3.68E−13 | 1.95E−12 | 1 |
| SARS-CoV-1 | | | | | | | |
| MERS-CoV | 4.10E−10 | 8.25E−11 | 4.96E−13 | 2.36E−13 | 3.76E−13 | 2.09E−12 | 1 |
| SARS-CoV-1 | | | | | | | |
| SARS-CoV-1 | 1.69E−04 | 4.71E−06 | 2.59E−07 | 1.23E−07 | 7.65E−08 | 1.33E−06 | 1 |
| SARS-CoV-2 | | | | | | | |
| Influenza A H1N1 | 0.42109 | 0.31383 | 0.14863 | 0.12069 | 0.10126 | 0.25589 | 0 |
| MERS-CoV | | | | | | | |
| Influenza A H1N1 | 0.46224 | 0.51144 | 0.23302 | 0.18526 | 0.11884 | 0.38013 | 0 |
| HCoV-229E | | | | | | | |
| MERS-CoV | 0.94208 | 0.71207 | 0.77357 | 0.78995 | 0.92318 | 0.78 | 0 |
| HCoV-229E | | | | | | | |
| F_{4, 10} values | 442.444 | 668.2656 | 1744.077 | 2025.4021 | 1896.4195 | 1293.194 | |

^{a}Significant p values are denoted by a one (1) and non-significant p values are denoted by a zero (0).

To investigate the versatility of the test response towards SARS-CoV-2, SPs from a series of its variants, Alpha B.1.1.7, Delta B.1.617.2, and Omicron B.1.1.529 were tested similarly on the original strain SP imprinted NMI/MIP assay, demonstrating an adaptable impedimetric response to identify the viral SP from different variants (FIG. 13d).

The selectivity of the NFluidEX towards SARS-CoV-2 IgG-RBD and IgM-RBD antibodies over those of other similar viruses was investigated. Similarly, the NMI/MIP test chambers imprinted SARS-CoV-2 IgG-RBD and IgM-RBD demonstrated higher impedimetric signals towards their targets with minimal cross-reactivity (FIG. 13b-13c). A null comparison between the results using one-way ANOVA with post hoc Holm-Sidak's test was performed. There was negligible cross-binding for Influenza A H1N1 IgG-N, Influenza A H1N1 IgG-RBD, HCoV-229E IgG-N, MERS-CoV IgG-N, MERS-CoV IgG-RBD, Influenza A H1N1 IgM-N, Influenza A H1N1 IgM-RBD, and MERS-CoV IgM-RBD in the SARS-CoV-2 IgG-RBD and IgM-RBD imprinted assays (p<0.001, Table 8-9. In particular, negligible cross-reactivity was detected for SARS-CoV-2 IgM-RBD on SARS-CoV-2 IgG-RBD assay and SARS-CoV-2 IgG-RBD on SARS-CoV-2 IgM-RBD assay (comparing the first and second columns in FIG. 13b-13c).

TABLE 8
A summary of statistical significance evaluation using a one-way ANOVA with post hoc
Holm-Sidak mean comparison test for serological selectivity of SARS-CoV-2 IgG-RBD

| | Blood p value | | Plasma p value | | Buffer p value | | |
|---|---|---|---|---|---|---|---|
| | 100 | 50 | 100 | 50 | 100 | 50 | |
| Mean Comparisons | pg · μl⁻¹ | pg · μl⁻¹ | pg · μl⁻¹ | pg · μl⁻¹ | pg · μl⁻¹ | pg · μl⁻¹ | Sig.^{a} |
| HCoV-229E (IgG-N) | 6.58E−15 | 6.25E−15 | 2.55E−14 | 3.30E−15 | 6.81E−14 | 3.43E−13 | 1 |
| SARS-CoV-2 (IgG- | | | | | | | |
| RBD) | | | | | | | |
| MERS-CoV (IgG-N) | 6.90E−15 | 6.33E−15 | 2.85E−14 | 3.63E−15 | 7.06E−14 | 3.67E−13 | 1 |
| SARS-CoV-2 (IgG- | | | | | | | |
| RBD) | | | | | | | |
| Influenza A H1N1 | 7.38E−15 | 6.51E−15 | 3.47E−14 | 3.85E−15 | 7.51E−14 | 3.99E−13 | 1 |
| (IgG-N) | | | | | | | |
| SARS-CoV-2 (IgG- | | | | | | | |
| RBD) | | | | | | | |
| MERS-CoV (IgG-RBD) | 7.51E−15 | 6.88E−15 | 3.94E−14 | 4.04E−15 | 8.30E−14 | 5.06E−13 | 1 |
| SARS-CoV-2 (IgG- | | | | | | | |
| RBD) | | | | | | | |
| Influenza A H1N1 | 9.09E−15 | 6.98E−15 | 3.95E−14 | 4.39E−15 | 9.89E−14 | 5.90E−13 | 1 |
| (IgG-RBD) | | | | | | | |
| SARS-CoV-2 (IgG- | | | | | | | |
| RBD) | | | | | | | |
| SARS-CoV-2 (IgM- | 1.06E−14 | 1.94E−14 | 5.32E−14 | 6.75E−15 | 1.11E−13 | 6.92E−13 | 1 |
| RBD) | | | | | | | |
| SARS-CoV-2 (IgG- | | | | | | | |
| RBD) | | | | | | | |
| HCoV-229E (IgG-N) | 0.26238 | 0.01724 | 0.12903 | 0.09116 | 0.32972 | 0.21885 | 0 |
| SARS-CoV-2 (IgM- | | | | | | | |
| RBD) | | | | | | | |
| MERS-CoV (IgG-N) | 0.31235 | 0.01826 | 0.1937 | 0.13861 | 0.36607 | 0.26549 | 0 |
| SARS-CoV-2 (IgM- | | | | | | | |
| RBD) | | | | | | | |
| Influenza A H1N1 | 0.39124 | 0.02095 | 0.34805 | 0.17898 | 0.43301 | 0.33389 | 0 |
| (IgG-N) | | | | | | | |
| SARS-CoV-2 (IgM- | | | | | | | |
| RBD) | | | | | | | |
| MERS-CoV (IgG-RBD) | 0.41432 | 0.02725 | 0.352 | 0.21783 | 0.45325 | 0.33436 | 0 |
| SARS-CoV-2 (IgM- | | | | | | | |
| RBD) | | | | | | | |
| HCoV-229E (IgG-N) | 0.44137 | 0.02922 | 0.36776 | 0.2977 | 0.49796 | 0.39771 | 0 |
| Influenza A H1N1 | | | | | | | |
| (IgG-RBD) | | | | | | | |
| MERS-CoV (IgG-N) | 0.51213 | 0.78972 | 0.4815 | 0.47602 | 0.55969 | 0.48163 | 0 |
| Influenza A H1N1 | | | | | | | |
| (IgG-RBD) | | | | | | | |
| Influenza A H1N1 | 0.6185 | 0.8119 | 0.48639 | 0.60871 | 0.57842 | 0.48737 | 0 |
| (IgG-N) | | | | | | | |
| Influenza A H1N1 | | | | | | | |
| (IgG-RBD) | | | | | | | |
| MERS-CoV (IgG-RBD) | 0.64851 | 0.81726 | 0.50615 | 0.63247 | 0.68638 | 0.56102 | 0 |
| Influenza A H1N1 | | | | | | | |
| (IgG-RBD) | | | | | | | |
| Influenza A H1N1 | 0.71294 | 0.83964 | 0.52601 | 0.69575 | 0.72481 | 0.58141 | 0 |
| (IgG-RBD) | | | | | | | |
| SARS-CoV-2 (IgM- | | | | | | | |
| RBD) | | | | | | | |
| MERS-CoV (IgG-RBD) | 0.74885 | 0.86583 | 0.53116 | 0.74395 | 0.74143 | 0.66919 | 0 |
| HCoV-229E (IgG-N) | | | | | | | |
| HCoV-229E (IgG-N) | 0.78125 | 0.89397 | 0.67052 | 0.78343 | 0.81506 | 0.77794 | 0 |
| Influenza A H1N1 | | | | | | | |
| (IgG-N) | | | | | | | |
| MERS-CoV (IgG-N) | 0.83913 | 0.92197 | 0.77711 | 0.81117 | 0.8369 | 0.77869 | 0 |
| MERS-CoV (IgG-RBD) | | | | | | | |
| MERS-CoV (IgG-N) | 0.87256 | 0.94486 | 0.78326 | 0.83766 | 0.8424 | 0.7859 | 0 |
| Influenza A H1N1 | | | | | | | |
| (IgG-N) | | | | | | | |
| MERS-CoV (IgG-N) | 0.9064 | 0.97151 | 0.80788 | 0.87845 | 0.90086 | 0.87589 | 0 |
| HCoV-229E (IgG-N) | | | | | | | |
| F_{6, 14} values | 327.5460 | 329.3973 | 263.5958 | 359.1365 | 232.9898 | 181.5124 | |

^{a}Significant p values are denoted by a one (1) and non-significant p values are denoted by a zero (0).

TABLE 9
A summary of statistical significance evaluation using a one-way ANOVA with post hoc
Holm-Sidak mean comparison test for serological selectivity of SARS-CoV-2 IgM-RBD

| | Blood p value | | Plasma p value | | Buffer p value | | |
|---|---|---|---|---|---|---|---|
| | 100 | 50 | 100 | 50 | 100 | 50 | |
| Mean Comparisons | pg · μl⁻¹ | pg · μl⁻¹ | pg · μl⁻¹ | pg · μl⁻¹ | pg · μl⁻¹ | pg · μl⁻¹ | Sig.^{a} |
| HCoV-229E (IgG-N) | 2.27E−12 | 1.68E−12 | 3.60E−13 | 4.12E−12 | 4.04E−11 | 1.09E−13 | 1 |
| SARS-CoV-2 (IgM- | | | | | | | |
| RBD) | | | | | | | |
| SARS-CoV-2 (IgM- | 2.70E−12 | 2.23E−12 | 3.61E−13 | 5.59E−12 | 4.06E−11 | 1.12E−13 | 1 |
| RBD) SARS-CoV-2 | | | | | | | |
| (IgG-RBD) | | | | | | | |
| Influenza A H1N1 | 3.09E−12 | 2.72E−12 | 3.65E−13 | 5.70E−12 | 4.57E−11 | 1.20E−13 | 1 |
| (IgM-N) | | | | | | | |
| SARS-CoV-2 (IgM- | | | | | | | |
| RBD) | | | | | | | |
| Influenza A H1N1 | 3.74E−12 | 3.01E−12 | 4.03E−13 | 5.89E−12 | 4.72E−11 | 1.21E−13 | 1 |
| (IgM-RBD) | | | | | | | |
| SARS-CoV-2 (IgM- | | | | | | | |
| RBD) | | | | | | | |
| MERS-CoV (IgM-RBD) | 3.82E−12 | 3.42E−12 | 4.57E−13 | 8.06E−12 | 5.77E−11 | 1.21E−13 | 1 |
| SARS-CoV-2 (IgM- | | | | | | | |
| RBD) | | | | | | | |
| MERS-CoV (IgM-RBD) | 0.2437 | 0.11387 | 0.52198 | 0.16223 | 0.51845 | 0.74855 | 0 |
| HCoV-229E (IgG-N) | | | | | | | |
| HCoV-229E (IgG-N) | 0.26263 | 0.18509 | 0.52751 | 0.43708 | 0.52297 | 0.75508 | 0 |
| Influenza A H1N1 | | | | | | | |
| (IgM-RBD) | | | | | | | |
| MERS-CoV (IgM-RBD) | 0.43195 | 0.26597 | 0.54373 | 0.43877 | 0.67162 | 0.77592 | 0 |
| SARS-CoV-2 (IgG- | | | | | | | |
| RBD) | | | | | | | |
| Influenza A H1N1 | 0.46046 | 0.33243 | 0.73458 | 0.46137 | 0.7158 | 0.81735 | 0 |
| (IgM-RBD) | | | | | | | |
| SARS-CoV-2 (IgG- | | | | | | | |
| RBD) | | | | | | | |
| HCoV-229E (IgG-N) | 0.47905 | 0.49084 | 0.75998 | 0.48041 | 0.77491 | 0.82406 | 0 |
| Influenza A H1N1 | | | | | | | |
| (IgM-N) | | | | | | | |
| MERS-CoV (IgM-RBD) | 0.62924 | 0.50013 | 0.76659 | 0.50431 | 0.78036 | 0.84544 | 0 |
| Influenza A H1N1 | | | | | | | |
| (IgM-N) | | | | | | | |
| Influenza A H1N1 | 0.66438 | 0.60008 | 0.78583 | 0.50615 | 0.82139 | 0.92827 | 0 |
| (IgM-N) | | | | | | | |
| Influenza A H1N1 | | | | | | | |
| (IgM-RBD) | | | | | | | |
| HCoV-229E (IgG-N) | 0.687 | 0.64575 | 0.97291 | 0.90993 | 0.82694 | 0.97118 | 0 |
| SARS-CoV-2 (IgG- | | | | | | | |
| RBD) | | | | | | | |
| Influenza A H1N1 | 0.75614 | 0.76985 | 0.97986 | 0.94332 | 0.9518 | 0.97808 | 0 |
| (IgM-N) | | | | | | | |
| SARS-CoV-2 (IgG- | | | | | | | |
| RBD) | | | | | | | |
| MERS-CoV (IgM-RBD) | 0.96045 | 0.81497 | 0.99304 | 0.96646 | 0.99429 | 0.9931 | 0 |
| Influenza A H1N1 | | | | | | | |
| (IgM-RBD) | | | | | | | |
| F_{5, 12} values | 255.7395 | 264.3684 | 361.9643 | 230.0317 | 161.3457 | 442.9206 | |

^{a}Significant p values are denoted by a one (1) and non-significant p values are denoted by a zero (0).

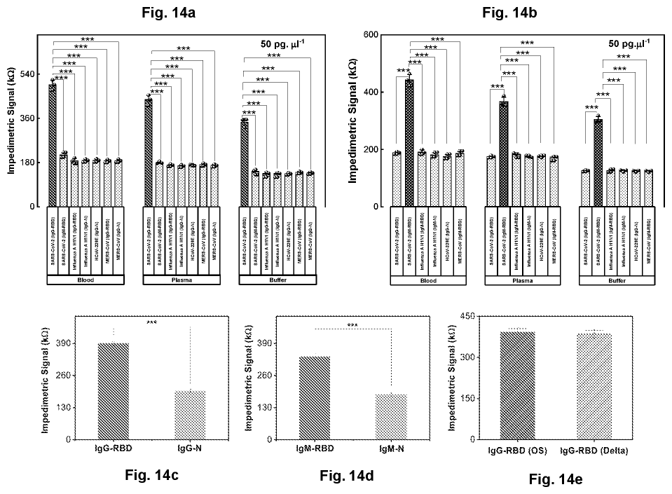

The selectivity of the assay towards the imprinted target was further tested with a determined concentration of the target and analogous viral particles within the linear range response of the assay, demonstrating a high selectivity at even lower concentrations (FIG. 14). In a comparative study, to confirm the higher accuracy in the readout when targeting the anti-RBD antibodies versus anti-nucleocapsid antibodies (IgG-N and IgM-N), the IgG-RBD and IgM-RBD entities were imprinted in the assay and tested with similar conditions (FIG. 14c, 14d). As predicted, an attenuated positive impedimetric signal was observed for IgG-N and IgM-N antibodies compared to the statistically higher response from anti-RBD antibodies (p<0.001, details in Supporting Information). The anti-RBD assay was tested with Delta B.1.617.2 anti-RBD antibodies (FIG. 14e and a nearly identical impedimetric signal was obtained, indicating that the original strain assay can be used to detect antibodies belonging to VOCs.

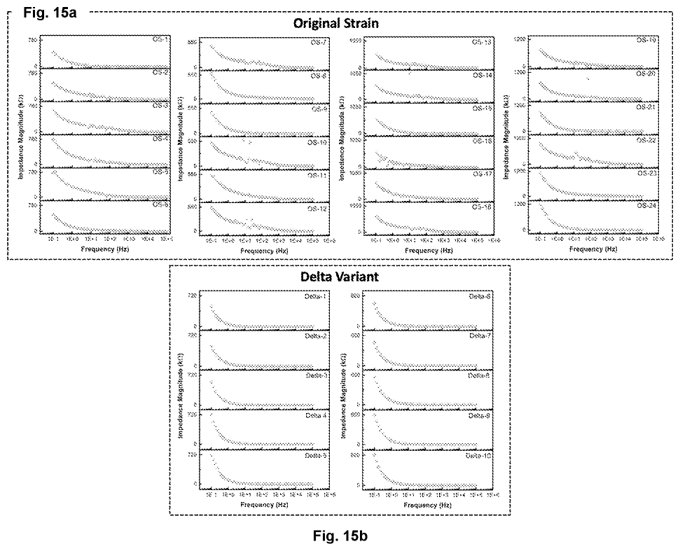

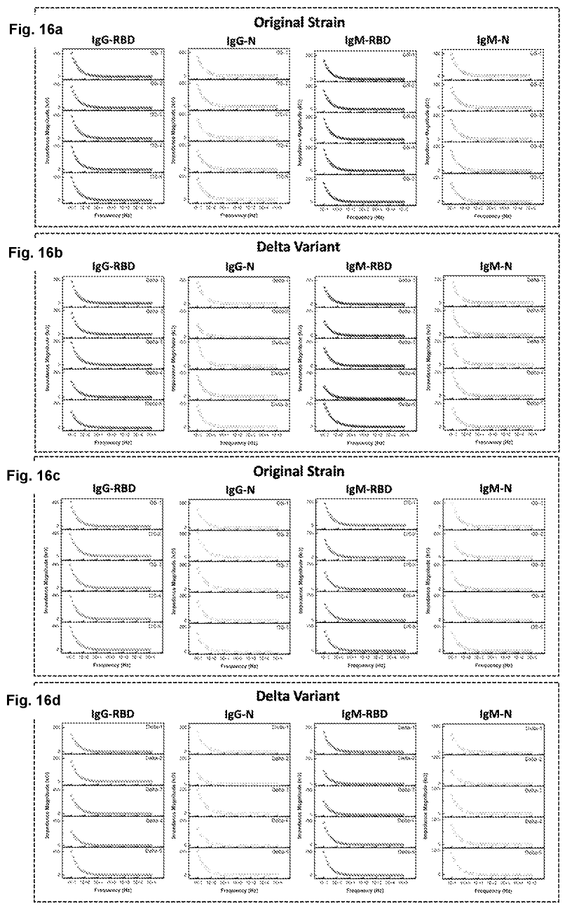

To demonstrate the applicability of the NFluidEX for clinical decision making, 34 COVID-19-positive saliva samples were analysed in contrast to 17 COVID-19-negative saliva samples, while simultaneously testing 10 COVID-19-positive patient blood samples in contrast to 8 COVID-19-negative blood samples for multiplexed serosurveillance of IgG-RBD and IgM-RBD antibodies (FIG. 15-16).

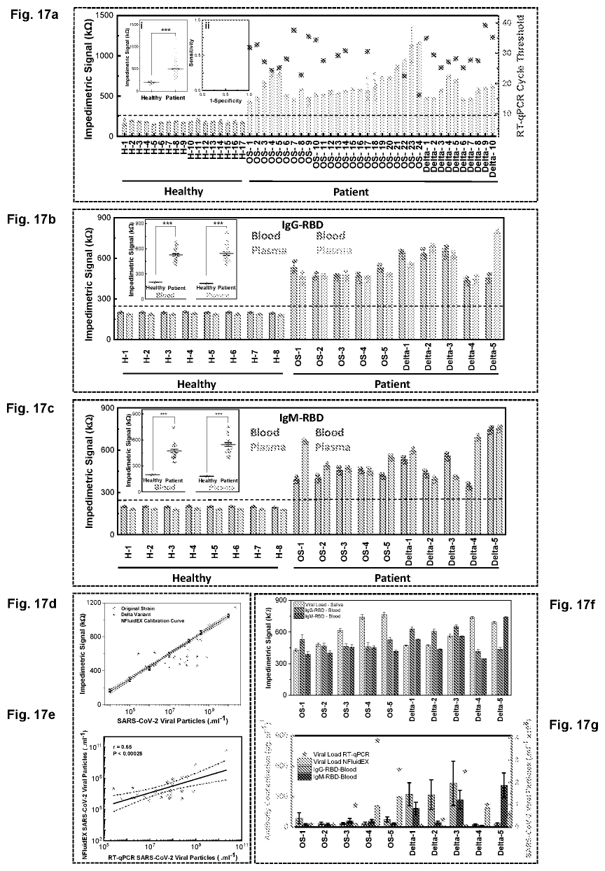

A set of randomized samples belonging to the SARS-CoV-2 original strain and the Delta B.1.617.2 variant was analysed. In order to quantifiably assess the impedimetric signal of NFluidEX towards SARS-CoV-2 viral concentration, the signals from a cohort of healthy samples (n=17) were compared to the signal from a cohort of patient samples (n=34) clinically diagnosed with SARS-CoV-2 original strain and Delta variant (FIG. 17a). A clear threshold to identify the SARS-CoV-2 viral infection based on the impedimetric signal in the patient samples was defined at 250 kΩ regardless of the viral infection strain. A statistically significant difference is achieved to differentiate between COVID-19-positive patient samples and healthy saliva samples (FIG. 17a, inset (i)), demonstrating a 100% sensing efficacy indicated via a receiver operating characteristic (ROC) curve (FIG. 17a, inset (ii)). COVID-19 positive blood and plasma samples (n=10) of patients were also tested with NFluidEX and compared them to healthy samples (n=8). A threshold impedimetric level clearly differentiates between positive patient signals and healthy signals both in blood and plasma for IgG-RBD and IgM-RBD antibodies (FIG. 17b-c). The post hoc comparisons via Holm-Sidak's test demonstrated a statistically significant difference (p<0.001) between the impedimetric signal of the positive patient samples versus negative patient samples both for blood and plasma, for IgG-RBD and IgM-RBD antibodies (FIG. 17b-c, inset). Table 10 summarizes the performance of the multiplexed NFluidEX test assay in comparison with the results reported by RT-qPCR and ELISA, where the overall parallel tests demonstrate 100% sensitivity and 100% specificity.

TABLE 10
Summary of NFluidEX performance against
current gold standard testing methods

| | | RT-qPCR | | |
|---|---|---|---|---|
| | | + | − | Total |
| NFluidEX | + | 34 | 0 | 34 |
| | − | 0 | 17 | 17 |
| | Overall result | 34 | 17 | 51 |
| | of NFluidEX | | | |

| | | | ELISA | | | | |
|---|---|---|---|---|---|---|---|
| | | | IgG | IgM | IgG | IgM | |
| | | | + | + | − | − | Total |
| NFluidEX | IgG | + | 10 | 0 | 1 | 0 | 11 |
| | IgM | + | 0 | 10 | 0 | 1 | 11 |
| | IgG | − | 0 | 0 | 7 | 0 | 7 |
| | IgM | − | 0 | 0 | 0 | 7 | 7 |

| | | | |
|---|---|---|---|
| Overall result | + | 10 | 0 |
| of NFluidEX | − | 0 | 8 |

+: positive test result,
−: negative test result;
Note:
Although a single false positive was recorded for IgG and IgM, the combined parallel sensitivity and specificity evaluated at two unique test sites yielded 100% accordance with gold standard methods.

To assess the quantitative nature of the NFluidEX compared to RT-qPCR, the viral load distribution in the patient samples was calculated based on both methods. For the gold standard RT-qPCR method, cycle threshold (Ct) values were obtained for all patient samples regardless of their viral strain, according to the established inversely proportional scaling between Ct values and viral loads. When the estimated viral load distribution of the patient samples was compared with the NFluidEX impedimetric calibration curve (FIG. 17d), a significant correlation between the tested linear range of the test assay and viral load distribution of the patient samples was observed. The distribution of viral load in patient samples estimated based on NFluidEX was studied as a function of estimated viral load based on the RT-qPCR test. The predicted viral content was strongly correlated in the 34 saliva samples that tested positive by both the NFluidEX device and conventional RT-qPCR analysis (FIG. 17e), exhibiting similar mean values (1.87×10⁹ and 0.96×10⁹ number of viral particles. ml⁻¹), with significant correlation. Discrepancies between the sensor provided herewith and the RT-qPCR results can be attributed to the 30-40% miss rate of RT-qPCR due to possible poor sample extraction and processing, in addition to challenges in RT-qPCR sensitivity when amplifying the RdRp, ORF1 ab, and N genes. However, the quantitative NFluidEX test with a low rate of error defines its potential as a reliable testing method compared to the current gold standard methods.

As a proof-of-concept to demonstrate the potential of the NFluidEX as provided herewith in the synchronous usage of saliva-based diagnosis and blood-based serology testing, a field study was conducted for a cohort of 10 patients (n=5 patients clinically diagnosed with the SARS-CoV-2 original strain and n=5 patients clinically diagnosed with the SARS-CoV-2 Delta B.1.617.2 variant). The dual detection device allowed for an enhanced combined sensitivity over diverse disease manifestations due to higher positive rates of diagnostic tests during the acute phase of infection and high positive rates of serology biomarkers during the convalescent phase of infection. All the patients in this cohort were evaluated 1 week after symptom onset. Regardless of their viral strain, all patients demonstrated positive results for both the NFluidEX saliva-diagnostic and blood-serosurveillance tests, which were in accordance with their reported RT-qPCR and ELISA results (FIG. 17f). Quantifiable multiplexed signal of NFluidEX monitored the diverse response of the individual patient samples with respect to the viral load and antibody concentration (FIG. 17g). It demonstrated that patients in the acute phase of infection displayed higher viral loads and low antibody production (OS-4, OS-5, Delta-4), while patients in the convalescent phase of infection displayed higher antibody levels and lower viral loads (Delta-1, Delta-2, Delta-3). Some patients displayed higher relative IgM-RBD content (Delta-5), which is indicative of developing immunity against the viral antigens, while others were still likely in an early-stages of infection (OS-1, OS-2, OS-3). This study confirmed the potential benefits of quantifiable monitoring outcome of heterogeneous disease dynamics that vary on the individual level.

To demonstrate the broad applicability of the NFluidEX test assay, it is demonstrated that the biomimic NMI/MIP assay is amenable to other contagious respiratory infections, like the Influenza A H1N1 virus. The assay was imprinted following the same protocol with the viral haemagglutinin surface protein of Influenza. Electrochemical sensing demonstrated a linearly increasing impedance magnitude over the varied concentration of viral protein. A similar linear trend over a wide linear range from 10 pg. ml⁻¹-10⁵ pg. ml⁻¹ at a low LOD was observed in both buffer and saliva (FIG. 19a-c). To validate the haemagglutinin imprinted geometric sites for the detection of whole Influenza viral particles, we challenged the assay with heat-inactivated viral particles, and observed linearly increasing impedimetric signal from 6.44×10⁶-2.58×10⁹ number of viral particles.ml⁻¹ at a low LOD in buffer and saliva (FIG. 19d-f).

Finally, an automatic readout system for nonprofessional users was developed. The assembly of the MIP assay with the devolved readout system provides a platform for rapid test, analysis, and monitoring of SARS-CoV-2 in real samples in 10 min or 1 min depending on the media. A smartphone gadget is developed to obtain an Arduino kit's sent or portable impedimetric signal transduction module signals, analyze, and monitor the risk level based on the received signals (FIG. 21a). The gadget is developed in an uncomplicated way that a user can automatically receive the data by a simple click on a provided button (FIG. 21c, 21e) and switch/control to select between whole virus or the simultaneous serological detection. The data is analyzed, and the electrochemical signal is measured automatically (FIG. 21e, 21e). Also encompassed is a portable custom made potentionstat.

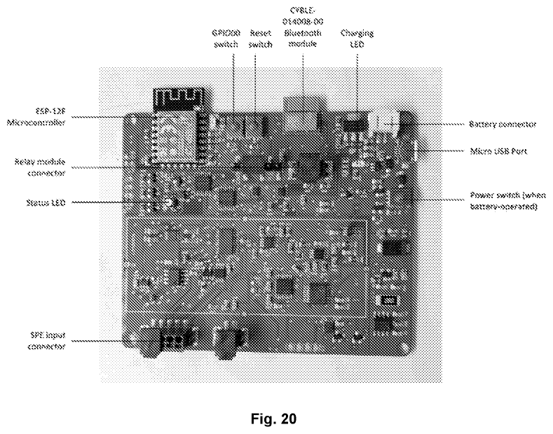

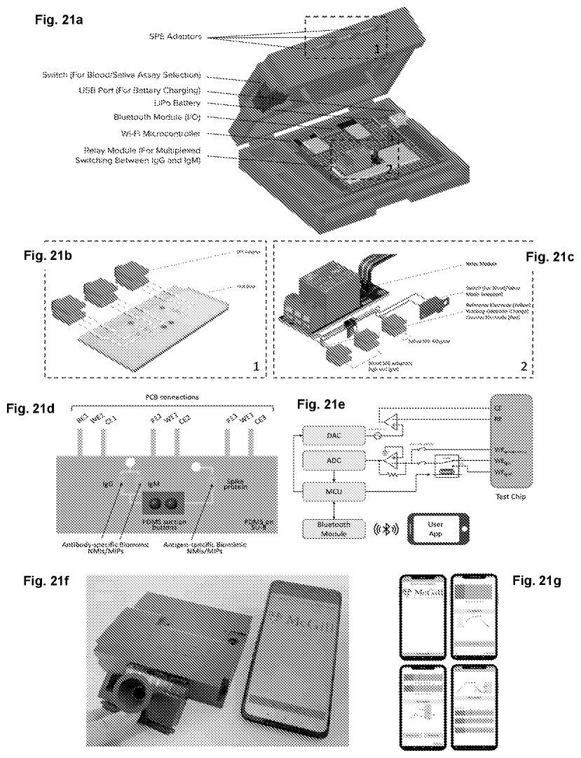

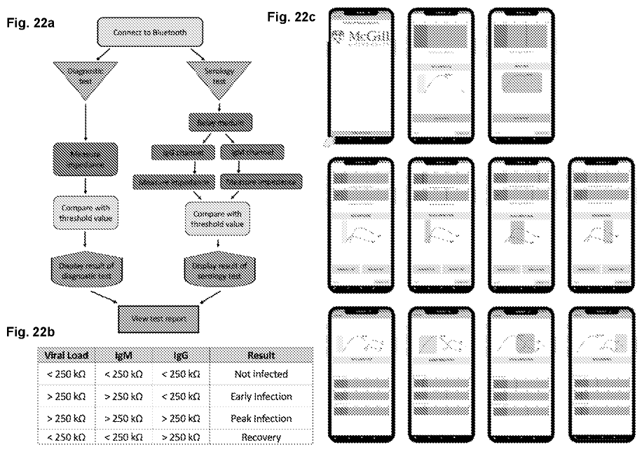

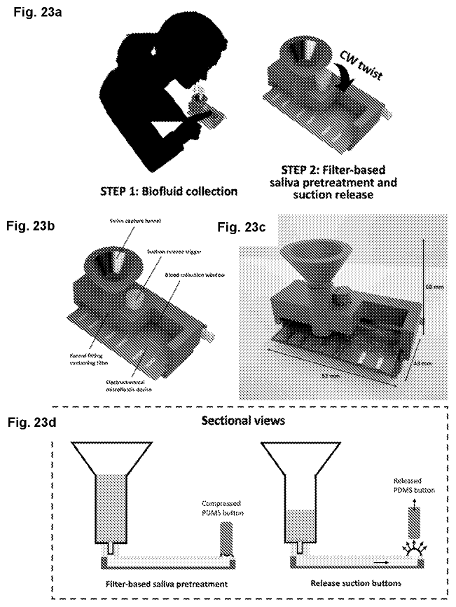

The signal transduction panel, potentionstat, is a portable cost-effective battery-operated electrochemical workstation that consists of a printed electrical circuit board and a Bluetooth wireless connection to convert the impedimetric signal of the test assay to a quantifiable readout on a smartphone via an Android application within 1 min (FIG. 20-23). A single-use sample collection cartridge embedded with a 3-channel multiplexed fluidic assay allows for easy self-collection of the saliva and blood by a lay-user (FIG. 23) which employs a filter-based technique that can remove large glycoproteins from the saliva while effectively reducing its viscosity with results comparable to that of from the centrifugation. This is being done via an integrated self-collection funnel that connects to the microfluidic device using custom 3D-printed attachments. A. Patient blood samples can be collected by a self-administered lancing device (˜3 μl) while patient saliva can be obtained via an attached self-collection funnel (˜700 μl). A bank of information is recorded in the gadget that correlates the current peak to a risk-level—impedimetric magnitude to the state and stage of infection—and assessment based on controlled sample measurement. By comparing the measured current peak with the recorded bank of information in the gadget, the risk-level or state is monitor in four main categories: Non infected, Early infection, Peak infection, and Recovery (FIG. 22). Using the smartphone gadget, first, the potentiostat/galvanostat cyclic voltammetry output is received by the Arduino kit or transduction module. The received signal is sent to the smartphone using a Wi-Fi micro-controller or BLE connector (FIG. 20). In FIG. 22b shown different displays of the application. The level of infection to SARS-CoV-2 is increasing when the measured current height is higher. While the probability of antibody in the sample is increasing when the measured current height is lower.

It is also encompass that the proposed biosensor allows detection of a protein of interest, not only viruses such as SARS-CoV2. As provided, the developed MIP biosensor offers sensitivity, stability, repeatability, and reproducibility towards protein detection.

The performance of the MIP biosensor was tested for detection of heart fatty acid binding protein (H-FABP) in clinical samples. Eight clinical blood serums samples were tested using commercial lateral flow assay LFA cassettes and the MIP biosensor (Table 2). Positive control (Pos. Ctrl) sample was HS-10D spiked with 100 ng mL⁻¹ H-FABP. The clinical samples without MI symptoms showed very low (i₀−i)/i₀ and tested negative H-FABP and Tnl on LFA cassettes (samples 1, and 2) which made them as negative control (Neg. Ctrl). The two samples (samples 3 and 8) tested negative H-FABP and positive Tnl on LFA cassettes, demonstrated low (i₀−i)/i₀ (less than 0.4) compared with the Pos. Ctrl while showed higher (i₀−i)/i₀ in comparison with the Neg. Ctrl. This implies that the level of H-FABP is lower than 8 ng mL⁻¹ (the LOD of the LFA for H-FABP, provided by the company) in these serums. Further, comparing these results with standard calibration plots of H-FABP in HS-10D, revealed that the levels of H-FABP in these samples are lower than 10 pg mL⁻¹, despite the negligible interference from Tnl. Sample 5 (tested negative for H-FABP and Tnl) showed higher (i₀−i)/i₀ (approximately 0.5). According to the calibration plot, the level of H-FABP in this sample is lower than 10 pg mL⁻¹, thus the LFA cassette cannot detect it. This clearly highlights the benefits of the developed biosensor with a lower LOD than the commercial device.

The other two samples (samples 4, and 7) that tested positive H-FABP on the LFA cassettes, displayed (i₀−i)/i₀ close to the Positive Control, confirming the ability of the device to reliably detect H-FABP in clinical samples with levels higher than 1 ng mL⁻¹. Sample 6 tested positive for H-FABP and Tnl by LFA. However, the (i₀−i)/i₀ recorded by the biosensor was less than 0.4, mainly related to the rather weak performance of the biosensor in high concentrated serum (the Tnl value for this sample was recorded more than 50000 ng mL⁻¹ according to the Table 2). Consequently, while the LFA needs expensive bioreceptors (antibodies), has a longer analysis time (˜10 minutes) and a high detection limit, the cost-effective MIP biosensor can successfully and specifically detect significantly lower levels of H-FABP in clinical samples, in less than 1 minute.

TABLE 11
The summary of the clinical samples investigation and comparison of the results
of the developed MIP biosensor with the LFA cassettes as a reference method

| Sample | Description of the | | | | H-FABP | |
|---|---|---|---|---|---|---|
| code | clinical sample* | LFA (TnI) | LFA (H-FABP) | (i₀ − i)/i₀ | value** | |
| Sample 1 | Patient without MI | Negative | Negative | 0.15 ± 0.02 | <<10 | pg mL⁻¹ |
| | symptoms | | | | | |
| Sample 2 | Patient without MI | Negative | Negative | 0.12 ± 0.05 | <<10 | pg mL⁻¹ |
| | symptoms | | | | | |
| Sample 3 | TnI~36.1 | Positive | Negative | 0.29 ± 0.04 | <10 | pg mL⁻¹ |
| Sample 4 | Negative troponin | Negative | Positive | 0.68 ± 0.05 | ~10 | ng mL⁻¹ |
| | but MI occurred | | | | | |
| Sample 5 | Negative troponin | Negative | Negative | 0.50 ± 0.05 | <10 | pg mL⁻¹ |
| | but MI occurred | | | | | |
| Sample 6 | TnI > 50000 | Positive | Positive | 0.36 ± 0.06 | <10 | pg mL⁻¹ |
| Sample 7 | Negative troponin | Negative | Positive | 0.70 ± 0.05 | ~30 | ng ml⁻¹ |
| | but MI occurred | | | | | |
| Sample 8 | TnI~27.7 | Positive | Negative | 0.26 ± 0.05 | <10 | pg mL⁻¹ |

*Troponin levels were the approximate values determined by ELISA at the hospital. MI occurrence was detected by chest pain and ST-segment elevation.
**The approximate values of H-FABP measured by the developed MIP biosensor. The values were determined through comparison of the recorded (i₀ − i)/i₀ for 10 times diluted clinical serum samples with the calibration plot of HS-10D spiked with various concentrations of H-FABP

In summary, the electrochemical biosensor provided based on a core-shell structure of NMIs and MIP allows for detection of protein such as e.g., but not limited to, H-FABP in PBS, human plasma, human serum, and bovine serum via a hybrid electrodeposition/electropolymerization fabrication protocol.

The proposed biosensor was used to detect H-FABP, as one of the early biomarkers of myocardial infarction with concentrations lower than 10 fg mL⁻¹ in PBS and in a short period of time (30 s). The biosensor showed lower LOD and wider linear range of detection than the commercial H-FABP LFA cassette. The selectivity evaluations also indicated distinguishable detection of H-FABP among other cardiac biomarkers, and in clinical samples owing to the biomimetic MIP layer. Moreover, the polymeric nature of the MIP layer provided high stability of the proposed biosensor at ambient temperature up to 3 weeks, depicting its excellent storage ability than the previously reported antibody-based biosensors that are usually stored at 4° C. As MIP biosensors are more stable, more efficient, and more scalable, than antibody-based biosensors at ambient temperature and in clinical environments, the disclosed biosensor combination provides for fast, sensitive, and affordable detection of proteins of interest.

### EXAMPLE I

### Device Fabrication and Testing

Polyaniline and ortho-phenylenediamine (o-PD) were bought from Thermo-fischer. Gold (III) chloride trihydrate was bought in SigmaAldrich. Heat inactivated SARS-CoV-2 (ATCC® VR1986HK™), SARS-CoV-2 Spike Antibody (CR3022) (NBP2-90980), SARS Nucleocapsid Protein Antibody (NB100-56683), and SARS Membrane Protein Antibody (NB100-56569) and Influenza A H1N1pdm (NY/01/09) Culture Fluid (Heat Inactivated) (0810248CFH1) purchased from Cedarlane. SARS-CoV-2 Nucleocapsid Antibody (N009) (NBP3-05721), Influenza A Haemagglutinin H1N1 Antibody (NBP3-06578) was bought from Novusbio. Pooled Saliva (IRHUSL50ML), Single Donor Human Plasma (Blood Derived) (IPLASK2E50ML) and Single Donor Human Whole blood (IWB1K2E10ML). Samples were bought from Innovative Research. Heat inactivated SARSCoV2 (ATCC® VR1986HK™) purchased from Cedarlane. Aniline 99.5%, Sodium Acetate ASC, Acetic Acid, and Phosphate buffer saline (PBS) 10× were bought in the chemical store of the Université du Quebec à Montreal. The chemicals purchased were analytical grade and were used without further purification. All the solutions were prepared using ultrapure water (>18 MΩ cm) from a Millipore Milli-Q water purification system.

### Solution Preparations

The gold solution for NMIs electrodeposition were prepared from Gold (III) chloride trihydrate (AuHCl) in 0.5 M HCl. The 10 mM electro-monomer ortho-phenylenediamine (o-PD) solution was prepared in acetate Buffer. Similarly, 10 mM, 20 mM, 50 mM, and 100 mM aniline electro-monomer solution was prepared in 0.5 M H₂SO₄. Washing solution, 0.1 M NaOH was prepared with ethanol and water in a 5:1 ratio. A 6.7 mM PBS (pH 7.2) containing 5 mM [Fe(CN)6]^{3-/4-} solution was prepared for electrochemical experiments.

### Saliva Collection Protocol

The human saliva samples were collected from healthy donors (2 female and 2 male) with an age range of 25-35 years old. The collection and processing protocols were adapted from Henson's and Alenus publications. Briefly, the donors were restrained from food and oral hygiene one-hour prior. A rinse and a pause step were followed prior the sample collection. The sample was processed through centrifuged at 10,000 rpm for 10 min at 4° C. Followed by separation of the fractions. Through the text, samples 1,2 and samples 3,4 resemble individual female and male human sample, respectively.

### Preparation of Viral Samples

Solutions spiked with heat inactivated SARS-CoV-2 virus (10⁵ pg. ml⁻¹) were prepared in PBS (pH 7.2) and human saliva. Followed by a series of 10-fold dilutions to cover a range of concentrations from (10 pg. ml⁻¹-10⁵ pg. ml⁻¹) in both media. Heat inactivated Influenza A H1N1 virus solutions were prepared equally. The solution spiked with antibodies against SARS-CoV-2 spike protein (1:100) were prepared in PBS (pH 7.2) and plasma (1:2). A serial of dilutions followed to cover different range of dilutions.

Device, MNIs and MIPs Fabrication

The device is based on a SU8 coated ITO-glass surface, where the analysis wells are patterned through standard lithography. Followed by a three-electrode electrodeposition method of gold to generate 3D hierarchical NMIs at the analysis wells base. The electrodeposition was preformed through an Autolab potentiostat/galvanostat (model: PGSTAT204), with a reference electrode of Ag/AgCl and a counter electrode of platinum wire. The supporting electrolyte solution, for the 3D gold NMIs were synthetization, was HAuCl₄ (1 mM) in a HCl (0.5 M). Synthesis was carried out at the applied fix potential of 600 mV vs Ag/AgCl. Electrosynthesis of polymer was carried out following a electropolymerization method. Briefly, a solution containing different concentrations of polymer (aniline, o-PD) was prepared using sodium acetate and H₂SO₄ solutions. SARS-CoV-2 heat-inactivated virus and antibody were added to the solution with a stock concentration and the volume of the monomer solution to the virus/antibody solution was maintained in the ratio of 95:5. Further, electropolymerization of polymer on NMIs electrode was carried out using cyclic voltammetry (CV) technique at a scan rate of 50 mV s⁻¹. Eventually, the samples were washed with ethanol and water solution (5:1 VN) containing 0.1 M NaOH to remove the template. Similarly, a control assay modified by non-imprinted polymer (NIP) was fabricated without using SARS-CoV-2 heat-inactivated virus or antibody as the template.

### Characterization

The morphology of the proposed sensor was study via scanning electron microscopy (SEM) images were captured with a Quanta FEG 450 ESEM (FE-SEM) and EIS characterization to assess the electrochemical performance of NMIs, NIP and MIP electrodes by using a 10 mM [Fe(CN)6]^{3-/4} PBS solution containing.

### COVID-19 Patient Sample Study

Human saliva, blood, and plasma samples were collected from the patients who were admitted at “Erythron Laboratory”, a cooperator laboratory of Isfahan University of Medical Sciences (IR.MUI.MED.REC.1400.066 and McGill IRB Internal Study Number: A03-M24-21B). Free authorization and consent forms were signed by patients, and their clinical samples were collected according to the laboratory regulation. 15 saliva samples were collected from adult patients with COVID-19 symptoms such as fever, fatigue, and dry cough were collected and tested with RT-qPCR (LightCycler 480, Roche) using primers (nCoV_IP2-12669Fw, nCoV_IP2-12759Rv, nCoV_IP2-12696bProbe(+)) targeting the RdRp gene/nCoV_IP2 in the ORF1ab prior to electrochemical studies. 5 samples were determined to belong to the original strain of SARS-CoV-2 and 10 samples belonged to the Delta B.1.617.2 variant. In addition, 19 patient saliva samples were collected from the University Health Network's PRESERVE-Pandemic Response Biobank for testing on the assay (REB #20-5364). All samples tested positively using RT-qPCR (QuantStudio 12K Flex, ThermoFisher) and were determined to be from the original strain of SARS-CoV-2 prior to electrochemical sensing. The samples were assessed at a Level 2+ facility situated in the Montreal Jewish General Hospital. In addition, 10 patient blood and plasma samples were collected for antibody evaluations of which 5 samples belonged to the original strain of SARS-CoV-2 and 5 samples belonged to the Delta B.1.617.2 variant. The blood samples were tested with ELISA reader (EUROIMMUN Analyzer I-2P). The ELISA results were presented as the cut-off index (COI) value for IgG-N, targeting nucleocapsid protein of SARS-CoV-2, and the sum of IgM-N and IgM-S, targeting nucleocapsid and spike proteins of SARS-CoV-2, respectively. Accordingly, the samples with a COI of higher than 1.1 and lower than 0.9 were considered as positive and negative samples, respectively.

### EXAMPLE II

### Electropolymerization of MIP-SARS-CoV-2 Heat-Inactivated Virus and Antibodies

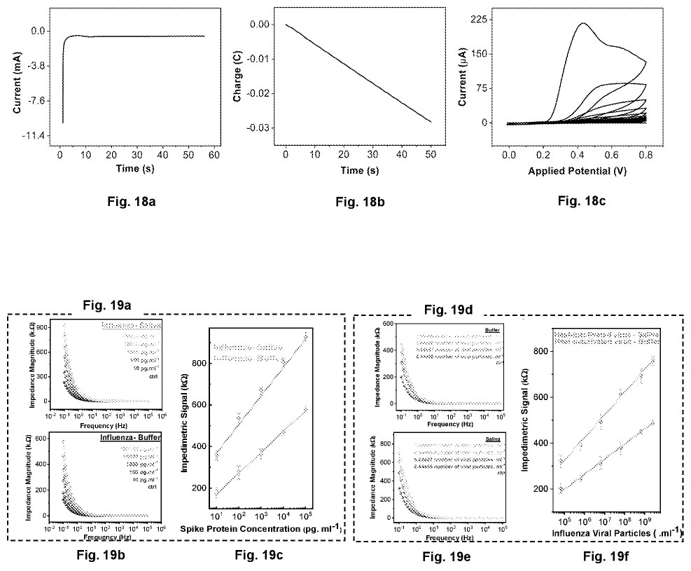

Chronoamperometry was performed to fabricate gold NMIs and cyclic voltammetry to electropolymerize nonconductive o-PD polymer to fabricate the MIP assay with various template proteins including the SARS-CoV-2 spike protein (SP) and anti-receptor binding domain (RBD) antibodies (IgG-RBD and IgM-RBD). (FIG. 18a) Gold NMIs electrodeposition using chronoamperometry, (FIG. 18b) corresponding charge on the working electrode during electrodeposition becoming more negative, (FIG. 18c) cyclic voltammetry for o-PD electropolymerization. 25 successive cycles of the electropolymerizing were done in the presence of template proteins on the NMIs surface. Two oxidation peaks are observed in the first cycle at about 0.4 and 0.7 V, which are related to the oxidation of o-PD; from the second to the tenth cycle, just one oxidation peak exists, which gradually shifts to more positive potentials, and its intensity decreases mainly due to the formation of a nonconductive layer on the surface. In the last cycle, the oxidation peaks of o-PD have completely disappeared, validating the creation of a continuous non-conductive layer on the surface.

The electrocatalytic performance of the NMI/MIPs electrode in real biological media was further studied. The SARS-CoV-2 heat-inactivated whole virus was spiked in human saliva. All set of experiments were conduncted as follow. First the incubation time needed to accurately detect the viral particle was determined by studying the charge transfer in pooled human saliva (FIG. 4a). Afterwards, the impedance responses of different concentrations of SARS-CoV-2 virus spiked in saliva and buffer was characterized (FIG. 8b).

The serological electrocatalytic performance of the NMI/MIPs electrode in real biological was assessed by the study of spike and nucleocapsid SARS-CoV-2 antibodies spiked in undiluted plasma and whole blood. The incubation time needed to accurately detect each antibody was determined in undiluted plasma for both IgG-RBD and IgM-RBD (FIG. 4b, 4c). The incubation period for the detection in whole blood was determined to be 1 min to prevent coagulation. Followed by the study of each antibody in spiked buffer and human samples (FIG. 11 and FIG. 12).

### EXAMPLE III

### Stability, Repeatability, and Reproducibility of the MIP Biosensor

One of the major merits of MIP biosensors is their high stability at ambient temperature. The stability of the MIP biosensor was examined during 21 days storing at ambient temperature. The R_{CT} for the MIP biosensor (virus and antibodies) was recorded after 7 days, and 21 days of storage in a shelf which demonstrated that the R_{CT} decrease by 2.5% and 5% after 7 and 21 days, respectively. These results signify that the proposed biosensor offers high stability at the ambient temperature. To ensure the absence of carryover effects upon successive measurements on a single sensor the repeatability of the biosensor was evaluated by the impedimetric repetitive measurements (five times, n=5). Each experiment was repeated for three individual electrodes and the relative standard deviation (RSD) was recorded 5.2%, which is in an acceptable range. Another important feature for practical applications of the biosensor is the reproducibility which was verified by recording the Rct for 5 as-prepared MIP electrodes, each one three times with a total RSD of 4.2% (n=5). A slight high value of this parameter can be related to the effective the distribution of the MIP layer and its thickness.

### EXAMPLE IV

### Digitalization

To digitize the detection process and use the built-in platform as a point-of-need device, a cyclic voltammetry technique (Electrochemical impedance spectroscopy) was used (FIG. 23). This technique is simpler and will take less time to process. After receiving signals from cyclic voltammetry from the impedimetric signal transduction module, where the signals are send to the application build-up as user interface. The module is connected to a Wi-Fi-enabled (i.e. Bluetooth module CYBLE-014008-00 Bluetooth module (Cypress, San Jose Calif., USA)) to send the signals to a smartphone. Android software is developed in Android Studio to analyze the received signals and correlate them to the stage of infection. In the transduction module, a A 1-channel relay module was connected to this microcontroller to allow for quasi-simultaneous EIS measurements between the IgG and IgM antibodies testing assays. The module converts the impedimetric signal of the test assay to a quantifiable readout on a smartphone via an Android application within 1 min. A bank of data with impedance magnitudes at a different stages of infection is recorded in the software based on the calibrated platform. The measured value based on received signals is compared with the bank of data. Finally, the infection stage is visualized in four stages: Non infected, Early infection, Peak infection, and Recovery

While the present disclosure has been described in connection with specific embodiments thereof, it will be understood that it is capable of further modifications and this application is intended to cover any variations, uses, or adaptations and including such departures from the present disclosure as come within known or customary practice within the art and as may be applied to the essential features hereinbefore set forth, and as follows in the scope of the appended claims.

What is claimed is:

1. A biosensor for detecting a target protein comprising:
a nano/micro islands (NMIs) core of gold spatially oriented with nanorough protrusions, and
a layer of electropolymerized molecularly imprinted polymers (MIP) polymerized on the NMIs core, said MIP consisting of a conductive monomer comprising a built-in recognition site of the target protein,
wherein the charge transfer resistance and/or impedance magnitude of the MIPs change upon binding of the target protein.

2. The biosensor of claim 1, wherein the NMIs are electrodeposited on a conductive glass with a reference electrode of Ag/AgCl and a counter electrode of platinum wire.

3. The biosensor of claim 2, wherein the conductive glass is a tin oxide (ITO) substrate.

4. The biosensor of claim 1, wherein conductive monomer is polyaniline (PANI) or o-phenylenediamine (o-PD).

5. The biosensor of claim 1, wherein the target protein is an antibody, a viral protein or a heart fatty acid binding protein (H-FABP).

6. The biosensor of claim 6, wherein the antibody is a viral antibody.

7. The biosensor of claim 5, wherein the viral protein is from SARS-CoV-2, severe acute respiratory syndrome coronavirus 1 (SARS-CoV-1), the human coronavirus 229E (HCoV-229E), the Middle East respiratory syndrome coronavirus (MERS-CoV), or Influenza.

8. The biosensor of claim 5, wherein the viral protein is from a SARS-CoV-2 variant.

9. A microfluidic read-out apparatus for detecting a target protein in a subject comprising:
i) the biosensor of claim 1; and
ii) microfluidic reader.

10. The apparatus of claim 9, wherein the microfluidic read-out apparatus is a multiplex microfluidic apparatus.

11. The apparatus of claim 9, further comprising a WiFi adapter for transferring the read-out signals from the microfluidic reader to a platform.

12. The apparatus of claim 11, wherein the WiFi adapter is a Bluetooth low energy (BLF) connector.13. The device of claim 9, wherein the platform is a computer or a smartphone.

14. A method of detecting a target protein in a subject comprising the steps of:
a) providing a sample from the subject;
b) contacting said sample with the biosensor of claim 1, wherein the presence of the target protein changes the charge transfer resistance and/or impedimetric of the MIPs upon binding of the target protein; and
c) transferring the change in charge transfer resistance signal and/or impedimetric signal to a microfluidic reader for transforming said signal into a cyclic voltammetry signal,
wherein the cyclic voltammetry signal and/or impedimetric signal indicates the presence of the target protein.

15. The method of claim 14, wherein the subject sample is a body fluid such as saliva, plasma, or whole blood.

16. The method of claim 14, wherein the subject is a human or an animal.

17. The method of claim 14, further comprising transmitting the cyclic voltammetry signal or impedimetric signal to a platform.

18. The method of claim 17, wherein the cyclic voltammetry signal or impedimetric signal is transmitted by Wi-Fi to the platform.

19. The method of claim 18, wherein the cyclic voltammetry signal or impedimetric signal is transmitted to a computer or a smartphone.

20. The method of claim 14, wherein the cyclic voltammetry signal indicates the presence of the target protein in 1 min to 11 min.
